(19) United States
(12) Patent Application Publication
(10) Pub. No.: US 2023/0235367 A1
(43) Pub. Date: Jul. 27, 2023

(54) PROCESS FOR PRODUCING ETHANOL FROM RAW STARCH USING ALPHA-AMYLASE VARIANTS

(71) Applicant: Novozymes A/S, Bagsvaerd (DK)

(72) Inventors: Carsten Andersen, Vaerloese (DK); Thomas Agersten Poulsen, Ballerup (DK); Dorota Nissen, Vedbaek (DK); Marie Thryøse Kruse, Vaerloese (DK); Sarah Schultheis Elliott, Raleigh, NC (US); Shiro Fukuyama, Chiba (JP)

(73) Assignee: Novozymes A/S, Bagsvaerd (DK)

(21) Appl. No.: 17/798,197

(22) PCT Filed: Feb. 9, 2021
(86) PCT No.: PCT/US2021/017178

§ 371 Date: Aug. 8, 2022

Related U.S. Application Data
(60) Provisional application No. 62/972,137, filed on Feb. 10, 2020.
(60) Provisional application No. 63/032,793, filed on Jun. 1, 2020.
(60) Provisional application No. 63/073,972, filed on Sep. 3, 2020.

(51) Int. Cl. C12P 7/10; C12N 9/26; C12N 15/81; C12N 15/52

(52) U.S. Cl. CPC C12P 7/10; C12N 9/2414; C12N 15/81; C12N 15/52; C12Y 302/01001

(57) ABSTRACT

The present invention relates to raw starch hydrolysis and in particular to a raw starch hydrolysis and fermentation process. More particularly the present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out using at least a variant alpha-amylase comprising a substitution at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to a process for producing ethanol from raw starch material by contacting the raw starch with a variant alpha-amylase during saccharification and fermentation.

### Description of the Related Art

Alpha-amylases (1,4-α-D-glucan glucanohydrolase, EC 3.2.1.1) constitute a group of enzymes which catalyze hydrolysis of starch and other linear and branched 1,4-glucosidic oligo- and polysaccharides.

Alpha-amylases are well known in industrial applications, e.g., in producing syrups or ethanol. One known alpha-amylase derived from Bacillus sp. belonging to the GH13_28 family is known to have some disadvantages for industrial applications because of poor stability at low pH, e.g., at pH below 5. There is therefore a need for improving pH stability of amylases belonging to the GH13_28 family, in order to improve the industrial applicability, e.g., in an ethanol production process from starch-containing material; either in a conventional starch to ethanol process or in a raw starch hydrolysis process.

Ethanol production from raw starch is normally performed as a one step process in which starch hydrolysis and fermentation is performed simultaneously, typically using an alpha-amylase and a glucoamylase to hydrolyze the raw starch and a yeast to ferment the glucose to produce ethanol. Process conditions are typically around 32° C. and at a pH from 4 to 5.
WO17/037614 (US2018265853 AA) discloses an alpha-amylase (SEQ ID NO: 6) having about 99% sequence identity to SEQ ID NO: 1 of the present disclosure.
US2019010473 discloses an alpha-amylase (SEQ ID NO: 34) having 87% sequence identity to SEQ ID NO: 1 of the present disclosure.
U.S. Pat. No. 9,090,887 BB discloses variants of an alpha-amylase (AmyE/SEQ ID NO: 2), wherein AmyE shares about 92% sequence identity with SEQ ID NO: 1 of the present disclosure.
WO17/133974 discloses an alpha-amylase (SEQ ID NO: 1) having about 97% sequence identity to SEQ ID NO: 1 of the present disclosure.
WO18/002360 discloses an alpha-amylase (SEQ ID NO: 2) having about 98% sequence identity to SEQ ID NO: 1 of the present disclosure.

The present invention provides a raw starch process wherein variant alpha-amylase having improved properties compared to its parent are applied during saccharification and fermentation.

### SUMMARY OF THE INVENTION

The present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

### BRIEF DESCRIPTION OF THE DRAWINGS

FIG. 1 shows the ethanol following 88 hours of fermentation by alpha-amylase treatment for Variant A and SEQ ID NO 1.

FIG. 2 shows the percent residual starch after 88 hours of fermentation by alpha-amylase treatment for Variant A and SEQ ID NO 1.

FIG. 3 shows the ethanol following 88 hours of fermentation by alpha-amylase treatment for Variant B, Variant C and SEQ ID NO 1.

FIG. 4 shows the g starch/g mash after 88 hours of fermentation by alpha-amylase treatment for Variant B, Variant C and SEQ ID NO 1.

FIG. 5 shows ethanol after 72 hours of fermentation for treatments with SEQ ID NO 1, Variant D and Variant E.

FIG. 6 shows ethanol after 72 h for fermentation for treatments with SEQ ID NO 1 and Variant F.

### DEFINITIONS

Alpha-amylase: Alpha-amylases (E.C. 3.2.1.1) are a group of enzymes which catalyze the hydrolysis of starch and other linear and branched 1,4 glucosidic oligo- and polysaccharides. The skilled person will know how to determine alpha-amylase activity. It may be determined according to the procedure described in the Examples, e.g., by measuring residual activity after stressing the sample at pH 4.0 using a commercial alpha-amylase activity assay kit, such as kits containing G7-pNP substrate and alpha-Glucosidase, e.g., manufactured by Roche/Hitachi (cat. No. 11876473) or Sigma-Aldrich (Catalog number MAK009).

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Carbohydrate Binding Module: The term “carbohydrate binding module” means a polypeptide amino acid sequence which binds preferentially to a poly- or oligosaccharide (carbohydrate), frequently—but not necessarily exclusively—to a water-insoluble (including crystalline) form thereof. A carbohydrate-binding module (CBM), is often referred to, a carbohydrate-binding domain (CBD).

CBMs derived from starch degrading enzymes are often referred to as starch-binding modules or SBMs (which may occur in certain amylolytic enzymes, such as certain glucoamylases (GA), or in enzymes such as cyclodextrin glucanotransferases, or in alpha-amylases). SBMs are often referred to as SBDs (Starch Binding Domains).

The parent alpha-amylase and the variant amylases to be applied in the process of invention preferably comprises a CBM, and in one embodiment the CBM comprises or consists of amino acids 527-626 of SEQ ID NO: 1.

Amino acids 439-526 of SEQ ID NO: 1 comprises or consists of a linker region.

In one embodiment the variant comprises a heterologous CBM, i.e., a CBM which is foreign (not naturally occurring in the parent wt amylase enzyme) to the parent alpha-amylase used as the starting point for the variants of the invention. Such a heterologous CBM is preferably a CBM of Family 20 or a CBM-20 module. Alternatively, the heterologous CBM can be selected from Carbohydrate-Binding Module Family 21, 25, 26, 34, 41 or 48.

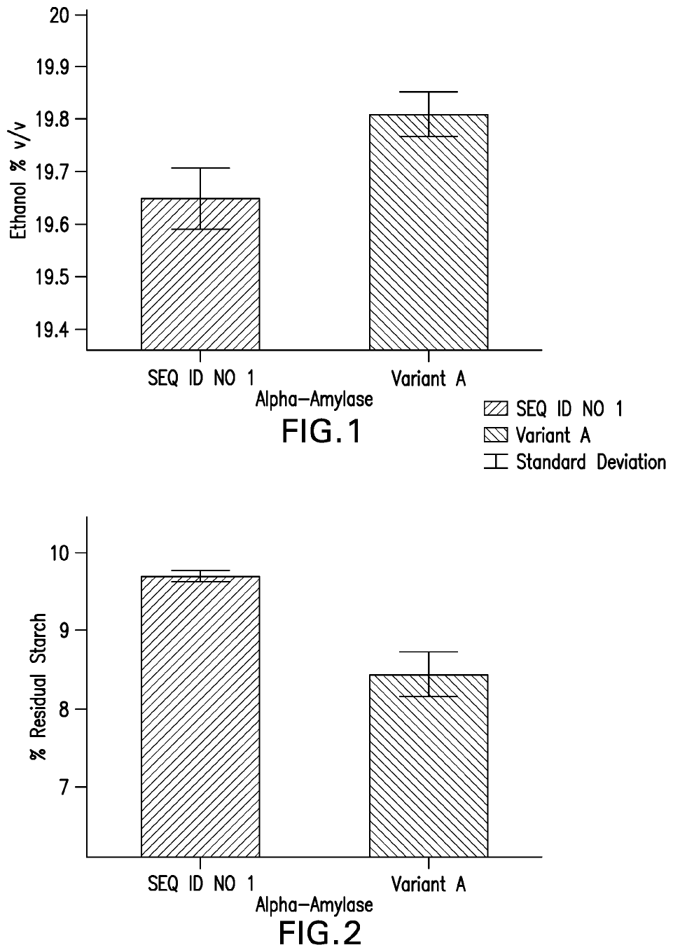

The “Carbohydrate-Binding Module of Family 20” or a CBM-20 module is in the context defined as a sequence of approximately 100 amino acids having at least 45% homology to the Carbohydrate-Binding Module (CBM) of the polypeptide disclosed in FIG. 1 by Joergensen et al. (1997) in Biotechnol. Lett. 19:1027-1031. The CBM comprises about 100 amino acids of the polypeptide, i.e., the subsequence from amino acid 582 to amino acid 683. The numbering of Glycoside Hydrolase Families applied in this disclosure follows the concept of Coutinho, P. M. & Henrissat, B. (1999) CAZy—Carbohydrate-Active Enzymes server at URL: http//afmb.cnrs-mrs.fr/˜-cazy/CAZY/index.html or alternatively Coutinho, P. M. & Henrissat, B. 1999; The modular structure of cellulases and other carbohydrate-active enzymes: an integrated database approach. In “Genetics, Biochemistry and Ecology of Cellulose Degradation”, K. Ohmiya, K. Hayashi, K. Sakka, Y. Kobayashi, S. Karita and T. Kimura eds., Uni Publishers Co., Tokyo, pp. 15-23 and Bourne, Y. & Henrissat, B. 2001; Glycoside hydrolases and glycosyltransferases: families and functional modules, Current Opinion in Structural Biology 11:593-600.

Examples of enzymes which comprise a CBM suitable for use in the context of the invention are alpha-amylases, maltogenic alpha-amylases, glucoamylases, beta-amylases, pullulanases, cellulases, xylanases, mannanases, arabinofuranosidases, acetylesterases and chitinases.

In one embodiment the CBM comprises or consists of amino acids 527-626 of SEQ ID NO: 1. This CBM belongs to Family 26 or CBM-26.

Catalytic domain: The term “catalytic domain” means the region of an enzyme containing the catalytic machinery of the enzyme. In one embodiment the catalytic domain comprises or consists of amino acids 12-438 of SEQ ID NO: 1.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has alpha-amylase activity. In one embodiment a fragment comprises or consists of amino acids 12-438 of SEQ ID NO: 1. In another embodiment a fragment comprises or consists of amino acids 1-438 of SEQ ID NO: 1.

Fusion polypeptide: The term “fusion polypeptide” is a polypeptide in which one polypeptide is fused at the N-terminus or the C-terminus of a variant of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Half-life: For a given alpha-amylase variant of the disclosure, the enzyme activity (measured as residual activity of a sample after incubation at pH 4.0 at 37° C. for 18-24 hours) of the stressed sample was divided by the enzyme activity of the unstressed sample, to compute residual activity (see examples for exact procedure). From this, the half-life in hours of the enzyme candidate is computed as the negative of the incubation-time in hours divided by log 2 of the residual activity.

Improvement Factor (IF): Improvement factor (IF) was calculated from the estimated half-life (T½), by dividing the estimated T½ for variants with the T½ of the wild type enzyme (SEQ ID NO:1).

Improved property: The term “improved property” means a characteristic associated with a variant that is improved, such as increased stability, compared to the parent. Such improved properties include, but are not limited to, pH stability. Increased pH stability may be determined as % residual activity of the variants according to the invention after stressing the enzyme by incubation at low pH, e.g., pH 4.0 at 37° C. C for 18-24 hours. Increased pH stability may also be determined as % residual activity of the variants according to the invention after stressing the enzyme by incubation at low pH, e.g., pH 4.0 at 32° C. for 24 or 96 hours. Residual activity determined for the variants of the invention may be determined as an improvement factor compared to the parent alpha-amylase of SEQ ID NO: 1.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Mature polypeptide: The term “mature polypeptide” means a polypeptide in its mature form following N-terminal processing (e.g., removal of signal peptide). In one embodiment the mature polypeptide is amino acids 1-626 of the polypeptide disclosed as SEQ ID NO: 1.

Mature polypeptide coding sequence: The term “mature polypeptide coding sequence” means a polynucleotide that encodes a mature polypeptide having alpha-amylase activity.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences. The one or more control sequences may be heterologous or foreign to the polynucleotide encoding the variant polypeptide of the invention.

Operably linked: The term “operably linked” means a configuration in which a control sequence is placed at an appropriate position relative to the coding sequence of a polynucleotide such that the control sequence directs expression of the coding sequence.

Parent or parent alpha-amylase: The term “parent” or “parent alpha-amylase” means an alpha-amylase to which an alteration is made to produce the enzyme variants of the present invention. The parent may be a naturally occurring (wild-type) polypeptide or a variant or fragment thereof.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

The sequence identity between two polynucleotide sequences can be determined using the same Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. The percent sequence identity is calculated as follows:

(Identical Deoxyribonucleotides×100)/(Length of the Alignment)

Subsequence: The term “subsequence” means a polynucleotide having one or more nucleotides absent from the 5′ and/or 3′ end of a mature polypeptide coding sequence; wherein the subsequence encodes a fragment having alpha-amylase activity.

Variant: The term “variant” means a polypeptide having alpha-amylase activity comprising an alteration, e.g., a substitution, an insertion, and/or a deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position The variants of the present invention have at least 20%, e.g., at least 40%, at least 50%, at least 60%, at least 70%, at least 80%, at least 90%, at least 95%, or at least 99% of the alpha-amylase activity of the parent alpha-amylase. The variant alpha-amylases according to the invention has increased stability at pH 4.0 compared to a parent alpha-amylase, and wherein increased pH stability at pH 4.0 can be determined as % residual alpha-amylase activity (% RA) after incubation of the variant amylase at pH 4.0, 32° C., for 18-24 hours. The Residual activity (RA %) after stress was calculated by dividing activity of stressed samples with unstressed samples and multiplying with 100. Alpha-amylase activity may e.g., be determined using the pNP-G7 alpha-amylase activity assay as described in the examples and in the material and methods section.

In one embodiment the parent alpha-amylase is preferably the polypeptide of SEQ ID NO: 1.

Raw Starch Material: The term “raw starch material” means primary starch based grains, which has not been subjected to temperatures above 57° C. for more than 10 minutes.

Raw Starch Hydrolysis (RSH): The term “raw starch hydrolysis” means the degradation of starch to polysaccharides from primary starch based grains which has not been subjected to temperatures above 57° C. for more than 10 minutes.

Raw Starch Hydrolysis and fermentation process: The term “raw starch hydrolysis and fermentation process” means the fermentation of starch to ethanol from primary starch based grains which has not been subjected to temperatures above 57° C. for more than 10 minutes.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants

For purposes of the present invention, the polypeptide disclosed in SEQ ID NO: 1 is used to determine the corresponding amino acid position in another alpha-amylase. The amino acid sequence of another alpha-amylase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Alterations as used herein includes substitutions, deletions and insertions as described below.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is described as: Original amino acid, position, original amino acid, inserted amino acid #1, inserted amino acid #2; etc. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to raw starch hydrolysis and in particular to a raw starch hydrolysis and fermentation process. More particularly the present invention relates to a process of producing a fermentation product, preferably ethanol, from raw starch material, preferably corn, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase of a parent alpha-amylase, wherein the variant comprises an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

The variant alpha-amylases applied according to the process of the invention are variant alpha-amylases having increased pH stability at acidic pH, such as at pH 4.0-5.5 compared to a parent alpha-amylase. The parent alpha-amylase is in one embodiment the mature polypeptide disclosed herein as SEQ ID NO: 1.

### Variants to be Applied in the Process of the Invention

The following sections disclose alpha-amylase variants to be used in the process of the invention.

In one particular embodiment, the variant alpha-amylases applied in the process of the invention are isolated alpha-amylase variants, comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1

In particular, the variant alpha-amylases applied in the process of the invention are isolated alpha-amylase variants, comprising a substitution at one or more positions corresponding to positions 64, 96, 150, 179, 196, 199, 207, 222, 284 and 603 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

In particular the alterations are selected from alterations selected from the group consisting of: E1*, T2*, A3*, N4*, K5*, S6*, N7*, K8*, V9*, V9D, V9L, T10*, T10I, A11*, S12*, 512P, S13*, V14*, V14I, K15*, N16*, N165, N28R, N28W, R38H, R38Y, D39R, A43D, A43T, A43V, K54I, G56P, G56W, N57P, R64S, Y67T, Y67W, W68S, W68Y, Y70F, Q71E, Q71N, Q86R, K89R, D90E, A94D, E96H, E96K, G99N, K101R, 1103Y, V107T, 1108L, 1108P, H110D, S113D, S113F, S113G, S113H, S113Q, S113W, S113Y, D114Q, A117T, N127D, Q134E, Q134L, Q134M, Q134N, Q134T, Q134W, W138Y, W142E, L150F, L150H, L150M, L150S, L150V, L150W, L150Y, G151F, G151S, G151W, G151Y, L152M, N156K, N156R, F169H, E171Q, L174I, D179G, D179S, Y183F, Y183I, D193S, D193DQ, D193DY, D193DQY, D193SQY, N196W, S199G, Q200W, N204D, 1205Y, N207W, T208N, T208S, S209L, F212W, L218F, L218W, S221N, A222E, A222I, A222V, R224K, N233S, H241N, S245N, H259Y, S275L, S275N, T278N, T278W, T278Y, N281Q, N281S, D282P, D283*, D283A, D283P, E284Q, E285V, T308M, T308Y, R323K, S335K, S335Q, S335R, T348K, E359Y, A382T, S386D, S388W, N392R, N392W, S394K, K396S, Q412W, A414K, K417W, K417Y, A424P, A428S, Q457L, Q457R, T459M, A466V, Q479QP, L489Q, E511D, G533H, Y534H, Q542K, V543P, A545P, 1547Y, K549*, K549Y, H550*, H550Y, D551*, G560P, A566P, N570H, M574MW, M574W, Y575W, T576Y, L577Y, T578Y, P580*, E581*, N582*, K589F, F592FK, V599W, N603W, P605S, D608Y, L614W, G619W, and H626* using SEQ ID NO: 1 for numbering or corresponding substitutions in another parent alpha-amylase.

In one embodiment the substitutions are selected from substitutions R64S, E96K, L150Y, L150W, L150H, L150M, L150F, D179S, N196W, S199G, N207W, A222E, A222I, A222V, E284Q and N603W using SEQ ID NO: 1 for numbering or corresponding substitutions in another parent alpha-amylase.

In case the parent alpha-amylase is different from SEQ ID NO: 1, the amino acid in a given position may be different from the amino acid present in the corresponding position in SEQ ID NO: 1. This, however, is understood to be within the scope of the present invention since the only essential feature is the amino acid in a given position after substitution.

In an aspect, the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent alpha-amylase.

In another aspect, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 1.

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises a substitution, at one or more positions corresponding to positions 64, 96, 150, 179, 196, 199, 207, 222, 284 and 603 of the polypeptide of SEQ ID NO: 1.

In one embodiment the substitutions are selected from substitutions R64S, E96K, L150Y, L150W, L150H, L150M, L150F, D179S, N196W, S199G, N207W, A222E, A222I, A222V, E284Q and N603W using SEQ ID NO: 1 for numbering or corresponding substitutions in another parent alpha-amylase.

In another aspect, the variant comprises or consists of a substitution at a position corresponding to position 196. In another aspect, the amino acid at a position corresponding to position 196 is substituted with Trp. In another aspect, the variant comprises or consists of the substitution N196W of the polypeptide of SEQ ID NO: 1.

In another aspect, the variant comprises or consists of a substitution at a position corresponding to position 199. In another aspect, the amino acid at a position corresponding to position 199 is substituted with Gly. In another aspect, the variant comprises or consists of the substitution S199G of the polypeptide of SEQ ID NO: 1.

In another aspect, the variant comprises or consists of a substitution at a position corresponding to position 222. In another aspect, the amino acid at a position corresponding to position 222 is substituted with Val. In another aspect, the variant comprises or consists of the substitution A222V of the polypeptide of SEQ ID NO: 1.

In another aspect, the variant comprises or consists of a substitution at a position corresponding to position 222. In another aspect, the amino acid at a position corresponding to position 222 is substituted with Ile. In another aspect, the variant comprises or consists of the substitution A222I of the polypeptide of SEQ ID NO: 1.

In another aspect, the variant comprises or consists of a substitution at a position corresponding to position 222. In another aspect, the amino acid at a position corresponding to position 222 is substituted with Glu. In another aspect, the variant comprises or consists of the substitution A222E of the polypeptide of SEQ ID NO: 1.

In another aspect, the variant comprises or consists of a substitution at a position corresponding to position 207. In another aspect, the amino acid at a position corresponding to position 207 is substituted with Trp. In another aspect, the variant comprises or consists of the substitution N207W of the polypeptide of SEQ ID NO: 1.

In another aspect, the variant comprises or consists of a substitution at a position corresponding to position 603. In another aspect, the amino acid at a position corresponding to position 603 is substituted with Trp. In another aspect, the variant comprises or consists of the substitution N603W of the polypeptide of SEQ ID NO: 1.

In another aspect, the variant comprises or consists of a substitution at a position corresponding to position 150. In another aspect, the amino acid at a position corresponding to position 150 is substituted with Tyr. In another aspect, the variant comprises or consists of the substitution L150Y of the polypeptide of SEQ ID NO: 1.

In another aspect, the variant comprises or consists of a substitution at a position corresponding to position 150. In another aspect, the amino acid at a position corresponding to position 150 is substituted with Trp. In another aspect, the variant comprises or consists of the substitution L150W of the polypeptide of SEQ ID NO: 1.

In another aspect, the variant comprises or consists of a substitution at a position corresponding to position 150. In another aspect, the amino acid at a position corresponding to position 150 is substituted with His. In another aspect, the variant comprises or consists of the substitution L150H of the polypeptide of SEQ ID NO: 1.

In another aspect, the variant comprises or consists of a substitution at a position corresponding to position 150. In another aspect, the amino acid at a position corresponding to position 150 is substituted with Met. In another aspect, the variant comprises or consists of the substitution L150M of the polypeptide of SEQ ID NO: 1.

In another aspect, the variant comprises or consists of a substitution at a position corresponding to position 150. In another aspect, the amino acid at a position corresponding to position 150 is substituted with Phe. In another aspect, the variant comprises or consists of the substitution L150F of the polypeptide of SEQ ID NO: 1.

In another aspect, the variant comprises or consists of a substitution at a position corresponding to position 64. In another aspect, the amino acid at a position corresponding to position 64 is substituted with Ser. In another aspect, the variant comprises or consists of the substitution R64S of the polypeptide of SEQ ID NO: 1.

In another aspect, the variant comprises or consists of a substitution at a position corresponding to position 96. In another aspect, the amino acid at a position corresponding to position 96 is substituted with Lys. In another aspect, the variant comprises or consists of the substitution E96K of the polypeptide of SEQ ID NO: 1.

In another aspect, the variant comprises or consists of a substitution at a position corresponding to position 179. In another aspect, the amino acid at a position corresponding to position 179 is substituted with Ser. In another aspect, the variant comprises or consists of the substitution D179S of the polypeptide of SEQ ID NO: 1.

In another aspect, the variant comprises or consists of a substitution at a position corresponding to position 284. In another aspect, the amino acid at a position corresponding to position 284 is substituted with Gln. In another aspect, the variant comprises or consists of the substitution E284Q of the polypeptide of SEQ ID NO: 1.

In another aspect, the variant comprises or consists of one or more substitutions selected from the group consisting of R64S, E96K, L150Y, L150W, L150H, L150M, L150F, D179S, N196W, S199G, N207W, A222E, A222I, A222V, E284Q and N603W using SEQ ID NO: 1 for numbering.

In one specific embodiment the present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out using at least a variant alpha-amylase comprising a substitution at one or more positions corresponding to positions 64, 96, 150, 179, 196, 199, 207, 222, 284 and 603 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

A more specific embodiment according to the invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out using at least a variant alpha-amylase comprising a substitution at one or more positions corresponding to positions 64, 96, 150, 179, 196, 199, 207, 222, 284 and 603 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase; wherein the variant alpha-amylase comprises a substitution or a combination of substitutions selected from:

### A222I;

### A222V;

### A222E;

### S199G;

### N196W;

### N207W;

### N603W:

### L150Y;

### L150W;

### L150H;

### L150M;

### L150F;

### R64S:

### E96K;

### D179S;

### E284Q;

### N207W+N603W;

### N196W+N207W;

### N196W+N603W;

### N196W+N207W+N603W;

### A222I+S199G+N196W;

### A222V+S199G+N196W;

### A222E+S199G+N196W;

### A222V+S199G+N196W+L150Y;

### L150H+S199G+A222I;

### L150M+S199G+A222V;

### N196W+S199G+A222V+N603W;

### L150F+N196W+S199G+A222I;

### L150M+N196W+S199G+A222V;

### L150W+S199G+A222V;

### L150H+N196W+S199G+A222V;

### L150W+N196W+S199G+A222I;L150Y+S199G+A222V;

### E96K+D179S+N196W+S199G+A222V+E284Q;

### R64S+E96K+N196W+S199G+A222V;

wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

It is well known in the art that possessing of the signal peptide may result in a distribution of different forms of mature polypeptides, thus in one embodiment the mature polypeptide will start from other positions than position 1 of SEQ ID NO: 1. E.g., the alpha-amylase variants of the present disclosure may comprise an N-terminal deletion, more particularly comprising at least amino acids 11 to 626 of SEQ ID NO: 1, at least amino acids 12 to 626 of SEQ ID NO: 1, such as at least amino acids 13 to 626 of SEQ ID NO: 1.

The variants of the disclosure may also comprise C-terminal deletions. E.g., in one embodiment the alpha-amylase variants of the disclosure comprise a C-terminal deletion, particularly H626*.

More particularly, the variant alpha-amylases, may comprise combinations of substitutions selected from:

### S199G+H626*;

### N196W+S199G+A222V+H626*;

### N196W+H626*

### N196W+N207W+H626*:

### L150Y+N196W+S199G+A222V

### E96K+D179S+N196W+S199G+A222V+E284Q;

### R64S+E96K+N196W+S199G+A222V;

and wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

In another specific embodiment the present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

In another specific embodiment the present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase, and wherein the variant alpha-amylase comprises an alteration or a combination of alterations selected from:

### N28W;

### 196W;

### S199G;

### N196W+V599W;

### N196W+H550Y+P605S;

### N196W+A545P+T576Y;

### N196W+K549Y+G560P;

### I108P+Y183I+N196W+1205Y;

### N196W+R323K;

### N196W+D283P;

### W138Y+N196W;

### L150W;

### N196W+N392W+K417W;

### N196W+N392R+K417W;

### N196W+K549*+H550*+D551*;

### N196W+P580*+E581*+N582*;

### N196W+F592FK;

### N28W+N196W+N207W+S386D+N603W;

### R38Y+N196W;

### N196W+H259Y;

### N196W+Q412W;

### N196W+F212W;

### N196W+V599W;

### N196W+H550Y+P605S;

### N196W+H550Y+K589F;

### N196W+H550Y+D608Y;

### N196W+M574W+L614W;

### N196W+G533H+M574W+L614W;

### N196W+V543P+N570H;

### N196W+G533H+Y575W+L614W;

### N196W+A545P+T576Y;

### N196W+A566P+T578Y;

### N196W+K549Y+G560P;

### N196W+A566P+L577Y;

### N196W+I547Y+G560P;

### N196W+M574MW;

### N196W+K549*+H550*+D551*+M574MW+P580*+E581*+N582*+F592FK;

### N196W+L614W+G619W;

### N28W+I108P+N196W+N207W+S386D+A466V+Q542K+N603W;

### N28W+I108P+N196W+N207W+S386D+N603W;

### N196W+D282P+D283*;

### W138Y+N196W;

### N28W+N196W+N207W+S386D+N603W;

### N196W+S388W+A424P;

### N196W+S388W+A424P+L489Q;

### A117T+N196W+H550Y+D608Y;

### N196W+Q457R+Y575W+L614W;

### N196W+S199G;

### N196W+A222V;

### N196W+A222E;

### N196W+A222I;

### S199G+A222V;

### S199G+A222E;

### S199G+A222I;

### N196W+S199G+A222I;

### Q134L;

### L150W+N156K+N196W+S199G+A222V;

### L150Y+N156K+N196W+S199G+A222I;

### L150W+N196W+S199G+A222I+A428S;

### L150F+N156K+N196W+S199G+A222I;

### L150Y+N156R+N196W+A222V;

### L150M+N156R+N196W+S199G+A222I;

### L150M+N156R+N196W+A222V;

### L150Y+N156R+N196W+S199G+A222V;

### L150M+N156K+N196W+S199G+A222V;

### L150Y+N156R+N196W+A222I;

### L150H+N156R+N196W+S199G+A222I;

### L150H+N156K+N196W+A222V;

### L150W+N156R+N196W+A222I;

### L150F+N156R+N196W+A222I;

### L150F+N156K+N196W+S199G+A222V;

### L150H+N156K+N196W+S199G+A222V;

### L150F+N156R+S199G+A222I;

### L150M+N156K+N196W+A222V;

### L150W+N156K+N196W+S199G+A222I;

### N156K+N196W+S199G+A222V;

### L150Y+N156R+S199G+A222I;

### L150M+N156R+S199G+A222I;

### L150W+N156K+S199G+A222V;

### L150W+N156R+N196W+S199G+A222V;

### L150Y+N156R+N196W;

### N156K+N196W+A222V;

### N156K+N196W+S199G;

### N156R+S199G+A222V;

### S113H+N196W+S199G+A222V;

### Q71E+S113H+N196W+S199G+A222V;

### N196W+S199G+A222V+D283A;

### N196W+S199G+A222V+D283P;

### W142E+D193SQY+N196W+S199G+A222V+R224K;

### E96K+K101R+L150W+N156R+D179S+N196W+S199G+T208N+A222V;

### E96K+K101R+L150Y+N156R+D179S+N196W+S199G+T208N+A222V+E284Q;

### E96K+K101R+L150M+N156R+D179S+N196W+S199G+T208N+A222V+E284Q;

### S113Q+Q134E+N196W+S199G+A222V;

### S113D+Q134N+N196W+S199G+A222V;

### S113F+N196W+S199G+A222V;

### E171Q+N196W+S199G+N204D+A222V;

### N196W+S199G+A222V+H241N+S245N+T278N+E284Q+E285V;

### N196W+S199G+A222V+S394K+A414K+K417Y;

### N196W+S199G+A222V+E359Y+S394K+K396S+A414K+K417Y;

### R38H+E96K+G99N+K101R+D179S+N196W+S199G+A222V;

### R38H+E96K+G99N+K101R+D179S+N196W+S199G+A222V+E284Q;

### V107T+H110D+N196W+S199G+A222V;

### Q134T+L150Y+N196W+S199G+A222V;

### S113F+L150W+N196W+S199G+A222V;

### S113F+L150Y+N196W+S199G+A222V;

### E96K+K101R+N156R+D179S+N196W+S199G+T208N+A222V;

### E96K+K101R+L150Y+N156R+D179S+N196W+S199G+T208N+A222V;

### E96K+K101R+L150M+N156R+D179S+N196W+S199G+T208N+A222V;

### E96K+K101R+N156R+D179S+N196W+S199G+T208N+A222V+E284Q;

### E96K+K101R+L150W+N156R+D179S+N196W+S199G+T208N+A222V+E284Q;

### N28R+Q86R+N196W+S199G+A222V;

### N28R+Q86R+K89R+N196W+S199G+A222V;

### G56P+N196W+S199G+S209L+A222V;

### E96K+N196W+S199G+A222V;

### T10I+N196W+S199G+A222V;

### D39R+N196W+S199G+A222V;

### R64S+N196W+S199G+A222V;

### T10I+D39R+R64S+N196W+S199G+A222V;

### T10I+D39R+N196W+S199G+A222V;

### D39R+E96K+N196W+S199G+A222V;

### R64S+D90E+E96K+N196W+S199G+A222V;

### R38H+D39R+E96K+G99N+K101R+D179S+N196W+S199G+A222V;

### R38H+R64S+E96K+G99N+K101R+D179S+N196W+S199G+A222V;

### T10I+R38H+R64S+E96K+G99N+K101R+D179S+N196W+S199G+A222V;

### T10I+R38H+R64S+D90E+E96K+G99N+K101R+D179S+N196W+S199G+A222V;

### T10I+R38H+D39R+E96K+G99N+K101R+D179S+N196W+S199G+A222V;

### R38H+D39R+R64S+E96K+G99N+K101R+D179S+N196W+S199G+A222V;

### E96K+N196W+S199G+A222V+E284Q;

### T10I+N196W+S199G+A222V+E284Q;

### D39R+N196W+S199G+A222V+E284Q;

### R64S+N196W+S199G+A222V+E284Q;

### T10I+D39R+E96K+N196W+S199G+A222V+E284Q;

### D193SQY+N196W+S199G+A222V;

### Q134T+N196W+S199G+A222V;

### L174I+N196W+S199G+T208N+A222V;

### Y183F+N196W+S199G+T208S+A222V;

### N127D+N156R+N196W+S199G+A222V;

### Q134T+L150W+N196W+S199G+A222V;

### N57P+N196W+S199G+A222V;

### N196W+S199G+Q200W+A222V;

### T10I+D39R+R64S+E96K+N196W+S199G+A222V+E284Q;

### T10I+D39R+R64S+N196W+S199G+A222V+E284Q;

### T10I+R64S+E96K+N196W+S199G+A222V+E284Q;

### D39R+R64S+D90E+E96K+N196W+S199G+A222V+E284Q;

### T10I+R64S+E96K+N196W+S199G+A222V;

### T10I+D39R+N196W+S199G+A222V+E284Q;

### D39R+E96K+N196W+S199G+A222V+E284Q;

### D39R+R64S+N196W+S199G+A222V+E284Q;

### R64S+E96K+N196W+S199G+A222V+E284Q;

### D39R+R64S+N196W+S199G+A222V;

### S12*+S13*+V14*+K15*+N16*+1103Y+N196W+S199G+A222V+N233S+T308Y;

### S12*+S13*+V14*+K15*+N16*+A43D+1103Y+N196W+S199G+A222V+N233S+T308M;

### V9L+S12P+V14I+N16S+A43T+N196W+S199G+A222V;

### S12*+S13*+V14*+K15*+N16*+N196W+S199G+A222V;

### N28W+N196W+S199G+A222V;

### N196W+S199G+A222V+N392W+K417W;

### T10I+D39R+E96K+N196W+S199G+A222V;

### V9D+R38H+N196W+S199G+A222V+T348K;

### S113F+L150Y+N156K+N196W+S199G+A222V;

### S113Y+L150Y+N156K+N196W+S199G+A222V;

### S113W+L150Y+N156K+N196W+S199G+A222V;

### S113F+N156K+N196W+S199G+A222V;

### S113Y+N156K+N196W+S199G+A222V;

### S113W+N156K+N196W+S199G+A222V;

### W138Y+L150V+N196W+S199G+A222V;

### W138Y+L150V+D179G+N196W+S199G+A222V;

### W138Y+L150V+N196W+S199G+L218W+A222V;

### E96K+Q134L+D179S+N196W+S199G+A222V+E284Q;

### E96K+Q134L+L150Y+N156R+D179S+N196W+S199G+A222V+E284Q;

### E96K+L150Y+N156R+D179S+N196W+S199G+A222V+E284Q;

### R38H+E96K+G99N+K101R+Q134L+D179S+N196W+S199G+S221N+A222V;

### R38H+E96K+G99N+K101R+Q134L+D179S+N196W+S199G+A222V;

### R38H+E96K+G99N+K101R+Q134L+L150Y+N156R+D179S+N196W+S199G+A222V;

### R38H+E96K+G99N+K101R+L150Y+N156R+D179S+N196W+S199G+A222V;

### L150F+N196W+S199G+A222I;

### Q134L+L150F+N156R+N196W+S199G+A222I;

### Q134L+L150H+N156R+N196W+S199G+A222I;

### Q134L+L150M+N156K+N196W+S199G+A222I;

### Q134L+L150Y+N156K+N196W+S199G+A222I+Q457L;

### Q134M+L150W+N156K+N196W+S199G+A222I;

### Q134W+L150W+N156K+N196W+S199G+A222I;

### L150W+L152M+N156K+N196W+S199G+A222I;

### S113F+L150W+N156K+N196W+S199G+A222I;

### S113Y+L150W+N156K+N196W+S199G+A222I;

### L150W+G151W+N156K+N196W+S199G+A222I;

### L150W+G151S+N156K+N196W+S199G+A222I;

### S113F+L150W+G151S+N156K+N196W+S199G+A222I;

### Y67W+W68Y+L150W+N156K+N196W+S199G+A222I;

### A43V+L150M+G151F+N156R+N196W+S199G+A222I;

### L150M+G151Y+N156R+N196W+S199G+A222I;

### L150M+G151W+N156R+N196W+S199G+A222I;

### L150M+G151S+N156R+N196W+S199G+A222I+Y534H;

### S113F+L150M+G151S+N156R+N196W+S199G+A222I;

### Q134L+L150F+N156K+N196W+S199G+A222I;

### L150W+G151F+N156K+N196W+S199G+A222I;

### L150W+G151Y+N156K+N196W+S199G+A222I;

### Y67W+W68Y+L150W+N156K+N196W+S199G+A222I;

### G56W+N57P+Y67W+W68Y+L150W+N156K+N196W+S199G+A222I;

### K54I+Y67W+W68S+S113G+N196W+S199G+A222V+A382T;

### K54I+Y67W+W68S+S113G+D114Q+L150V+N196W+S199G+A222V;

### K54I+Y67W+W68S+S113G+W138Y+N196W+S199G+A222V;

### K54I+Y67W+W68S+S113G+D114Q+W138Y+L150V+N196W+S199G+A222V;

### Q134L+N196W+S199G+A222V;

### V107T+I108L+H110D+F169H+N196W+S199G+A222V;

### N196W+S199G+A222V+N392W+K417W;

### V9D+R38H+N196W+S199G+A222V+T348K;

### E96H+L150Y+N156R+N196W+S199G+A222V+E284Q;

### Q134L+L150Y+N196W+S199G+A222V;

### L150Y+N196W+S199G+A222V;

### Q134L+L150Y+N196W+S199G+A222V;

### L150F+N156R+N196W+S199G+A222V;

### L150H+N156R+N196W+S199G+A222V;

### Q134L+L150F+N156R+N196W+S199G+A222V;

### Q134L+L150H+N156R+N196W+S199G+A222V;

### Q134L+L150F+N156K+N196W+S199G+A222V;

### Q134L+L150H+N156K+N196W+S199G+A222V;

### Q134L+L150Y+N156K+N196W+S199G+A222V;

### L150Y+N156K+N196W+S199G+A222V;

### Q134L+L150Y+N156K+N196W+S199G+A222V;

### Q134W+L150Y+N156K+N196W+S199G+A222V;

### Q134M+L150Y+N156K+N196W+S199G+A222V;

### Q134M+L150Y+N156K+N196W+S199G+A222V+A466V;

### L150Y+L152M+N156K+N196W+S199G+A222V;

### L150S+N196W+S199G+A222V;

### N196W+S199G+A222V+N281S;

### Y67T+N196W+S199G+A222V;

### Q71N+N196W+S199G+A222V;

### Q71N+A94D+N196W+S199G+A222V;

### N196W+S199G+L218F+A222V;

### N196W+S199G+L218W+A222V;

### N196W+S199G+A222V+T278W;

### N196W+S199G+A222V+T278W+T459M;

### N196W+S199G+A222V+T278Y;

### N196W+S199G+A222V+S275N;

### N196W+S199G+A222V+S275L;

### N196W+S199G+A222V+S335Q;

### N196W+S199G+A222V+S335K;

### N196W+S199G+A222V+S335R;

### N196W+S199G+L218W+A222V+S335K;

### N196W+S199G+L218W+A222V+S335Q;

### Y67W+N196W+S199G+A222V;

### N196W+S199G+A222V+N281Q;

### L150M+N156R+N196W+S199G+A222V;

### Q134L+L150M+N156R+N196W+S199G+A222V;

### Q134L+L150M+N156K+N196W+S199G+A222V;

### D39R+N196W+S199G+A222V+N281Q+E284Q;

### D39R+N196W+S199G+A222V+E284Q+Q479QP;

### D39R+Y70F+N196W+S199G+A222V+E284Q;

### N28W+D39R+N196W+S199G+A222V+E284Q;

### D39R+N196W+S199G+N207W+A222V+E284Q;

### E1*+T2*+A3*+N4*+K5*+S6*+N7*+K8*+D39R+N196W+S199G+N207W+A222V+E284Q;

### N196W+N207W;

### N196W+N207W+E284Q;

### E1*+T2*+A3*+N4*+K5*+S6*+N7*+K8*+N196W+N207W+E284Q;

### L150Y+N196W+S199G+A222V;

### E1*+T2*+A3*+N4*+K5*+S6*+N7*+K8*+L150Y+N196W+S199G+A222V;

### E96K+K101R+L150M+N156R+D179S+N196W+S199G+N207W+T208N+A222V;

### E1*+T2*+A3*+N4*+K5*+S6*+N7*+K8*+E96K+K101R+L150M+N156R+D179S+N196W+S199G+T208N+A222V;

### Q134L+L150H+N156R+N196W+S199G+A222I+E284Q;

### E1*+T2*+A3*+N4*+K5*+S6*+N7*+K8*+Q134L+L150H+N156R+N196W+S199G+A222I;

### E1*+T2*+A3*+N4*+K5*+S6*+N7*+K8*+Q134L+L150H+N156R+N196W+S199G+N207W+A222I+E284Q;

### E96K+D179S+N196W+S199G+A222V+E284Q;

### E1*+T2*+A3*+N4*+K5*+S6*+N7*+K8*+E96K+D179S+N196W+S199G+A222V+E284Q;

### R64S+E96K+N196W+S199G+N207W+A222V;

### N28W+D39R+N196W+S199G+N207W+A222V+E284Q;

### D39R+N196W+S199G+A222V+E284Q;

### D39R+N196W+S199G+N207W+A222V+E284Q;

### E1*+T2*+A3*+N4*+K5*+S6*+N7*+K8*+D39R+N196W+S199G+A222V+E284Q;

### E1*+T2*+A3*+N4*+K5*+S6*+N7*+K8*+N196W+N207W+E511D;

### L150Y+N196W+S199G+N207W+A222V;

### E96K+K101R+L150M+N156R+D179S+N196W+S199G+T208N+A222V;

### E96K+K101R+L150M+N156R+D179S+N196W+S199G+T208N+A222V+E284Q;

### E1*+T2*+A3*+N4*+K5*+S6*+N7*+K8*+E96K+K101R+L150M+N156R+D179S+N196W+S199G+N207W+T208N+A222V+E284Q;

### Q134L+L150H+N156R+N196W+S199G+A222I;

### N28W+E96K+D179S+N196W+S199G+A222V+E284Q;

### R64S+E96K+N196W+S199G+A222V;

### R64S+E96K+N196W+S199G+A222V+E284Q;

### E1*+T2*+A3*+N4*+K5*+S6*+N7*+K8*+R64S+E96K+N196W+S199G+A222V;

and wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

The variant alpha-amylases may further comprise C-terminal deletion, particularly H626*.

Thus, in another particular embodiments the present invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase, wherein the variant alpha-amylase comprises a combination of alterations selected from:

### N28W+H626*;

### S199G+H626*;

### N196W+V599W+H626*;

### N196W+H550Y+P605S+H626*;

### N196W+A545P+T576Y+H626*;

### N196W+K549Y+G560P+H626*;

### 1108P+Y183I+N196W+1205Y+H626*;

### N196W+R323K+H626*;

### N196W+D283P+H626*;

### W138Y+N196W+H626*;

### L150W+H626*;

### N196W+N392W+K417W+H626*;

### N196W+N392R+K417W+H626*;

### N196W+K549*+H550*+D551*+H626*;

### N196W+P580*+E581*+N582*+H626*;

### N196W+F592FK+H626*;

### N28W+N196W+N207W+S386D+N603W+H626*;

### R38Y+N196W+H626*;

### N196W+H259Y+H626*;

### N196W+Q412W+H626*;

### N196W+F212W+H626*;

### N196W+V599W+H626*;

### N196W+H550Y+P605S+H626*;

### N196W+H550Y+K589F+H626*;

### N196W+H550Y+D608Y+H626*;

### N196W+M574W+L614W+H626*;

### N196W+G533H+M574W+L614W+H626*;

### N196W+V543P+N570H+H626*;

### N196W+G533H+Y575W+L614W+H626*;

### N196W+A545P+T576Y+H626*;

### N196W+A566P+T578Y+H626*;

### N196W+K549Y+G560P+H626*;

### N196W+A566P+L577Y+H626*;

### N196W+I547Y+G560P+H626*;

### N196W+M574MW+H626*;

### N196W+K549*+H550*+D551*+M574MW+P580*+E581*+N582*+F592FK+H626*;

### N196W+L614W+G619W+H626*;

### N28W+I108P+N196W+N207W+S386D+A466V+Q542K+N603W+H626*;

### N28W+I108P+N196W+N207W+S386D+N603W+H626*;

### N196W+D282P+D283*+H626*;

### W138Y+N196W+H626*;

### N28W+N196W+N207W+S386D+N603W+H626*;

### N196W+S388W+A424P+H626*;

### N196W+S388W+A424P+L489Q+H626*;

### A117T+N196W+H550Y+D608Y+H626*;

### N196W+Q457R+Y575W+L614W+H626*;

### N196W+S199G;

### N196W+A222V;

### N196W+A222E;

### N196W+A222I;

### S199G+A222V;

### S199G+A222E;

### S199G+A222I;

### N196W+S199G+A222I;

### Q134L+H626*;

### L150W+N156K+N196W+S199G+A222V;

### L150Y+N156K+N196W+S199G+A222I;

### L150W+N196W+S199G+A222I+A428S;

### L150F+N156K+N196W+S199G+A222I;

### L150Y+N156R+N196W+A222V;

### L150M+N156R+N196W+S199G+A222I;

### L150M+N156R+N196W+A222V;

### L150Y+N156R+N196W+S199G+A222V;

### L150M+N156K+N196W+S199G+A222V;

### L150Y+N156R+N196W+A222I;

### L150H+N156R+N196W+S199G+A222I;

### L150H+N156K+N196W+A222V;

### L150W+N156R+N196W+A222I;

### L150F+N156R+N196W+A222I;

### L150F+N156K+N196W+S199G+A222V;

### L150H+N156K+N196W+S199G+A222V;

### L150F+N156R+S199G+A222I;

### L150M+N156K+N196W+A222V;

### L150W+N156K+N196W+S199G+A222I;

### N156K+N196W+S199G+A222V;

### L150Y+N156R+S199G+A222I;

### L150M+N156R+S199G+A222I;

### L150W+N156K+S199G+A222V;

### L150W+N156R+N196W+S199G+A222V;

### L150Y+N156R+N196W;

### N156K+N196W+A222V;

### N156K+N196W+S199G;

### N156R+S199G+A222V;

### S113H+N196W+S199G+A222V;

### Q71E+S113H+N196W+S199G+A222V;

### N196W+S199G+A222V+D283A;

### N196W+S199G+A222V+D283P;

### W142E+D193SQY+N196W+S199G+A222V+R224K;

### E96K+K101R+L150W+N156R+D179S+N196W+S199G+T208N+A222V;

### E96K+K101R+L150Y+N156R+D179S+N196W+S199G+T208N+A222V+E284Q;

### E96K+K101R+L150M+N156R+D179S+N196W+S199G+T208N+A222V+E284Q;

### S113Q+Q134E+N196W+S199G+A222V;

### S113D+Q134N+N196W+S199G+A222V;

### S113F+N196W+S199G+A222V;

### E171Q+N196W+S199G+N204D+A222V;

### N196W+S199G+A222V+H241N+S245N+T278N+E284Q+E285V;

### N196W+S199G+A222V+S394K+A414K+K417Y;

### N196W+S199G+A222V+E359Y+S394K+K396S+A414K+K417Y;

### R38H+E96K+G99N+K101R+D179S+N196W+S199G+A222V;

### R38H+E96K+G99N+K101R+D179S+N196W+S199G+A222V+E284Q;

### V107T+H110D+N196W+S199G+A222V;

### Q134T+L150Y+N196W+S199G+A222V;

### S113F+L150W+N196W+S199G+A222V;

### S113F+L150Y+N196W+S199G+A222V;

### E96K+K101R+N156R+D179S+N196W+S199G+T208N+A222V;

### E96K+K101R+L150Y+N156R+D179S+N196W+S199G+T208N+A222V;

### E96K+K101R+L150M+N156R+D179S+N196W+S199G+T208N+A222V;

### E96K+K101R+N156R+D179S+N196W+S199G+T208N+A222V+E284Q;

### E96K+K101R+L150W+N156R+D179S+N196W+S199G+T208N+A222V+E284Q;

### N28R+Q86R+N196W+S199G+A222V;

### N28R+Q86R+K89R+N196W+S199G+A222V;

### G56P+N196W+S199G+S209L+A222V;

### E96K+N196W+S199G+A222V;

### T10I+N196W+S199G+A222V;

### D39R+N196W+S199G+A222V;

### R64S+N196W+S199G+A222V;

### T10I+D39R+R64S+N196W+S199G+A222V;

### T10I+D39R+N196W+S199G+A222V;

### D39R+E96K+N196W+S199G+A222V;

### R64S+D90E+E96K+N196W+S199G+A222V;

### R38H+D39R+E96K+G99N+K101R+D179S+N196W+S199G+A222V;

### R38H+R64S+E96K+G99N+K101R+D179S+N196W+S199G+A222V;

### T10I+R38H+R64S+E96K+G99N+K101R+D179S+N196W+S199G+A222V;

### T10I+R38H+R64S+D90E+E96K+G99N+K101R+D179S+N196W+S199G+A222V;

### T10I+R38H+D39R+E96K+G99N+K101R+D179S+N196W+S199G+A222V;

### R38H+D39R+R64S+E96K+G99N+K101R+D179S+N196W+S199G+A222V;

### E96K+N196W+S199G+A222V+E284Q;

### T10I+N196W+S199G+A222V+E284Q;

### D39R+N196W+S199G+A222V+E284Q;

### R64S+N196W+S199G+A222V+E284Q;

### T10I+D39R+E96K+N196W+S199G+A222V+E284Q;

### D193SQY+N196W+S199G+A222V;

### Q134T+N196W+S199G+A222V;

### L174I+N196W+S199G+T208N+A222V;

### Y183F+N196W+S199G+T208S+A222V;

### N127D+N156R+N196W+S199G+A222V;

### Q134T+L150W+N196W+S199G+A222V;

### N57P+N196W+S199G+A222V;

### N196W+S199G+Q200W+A222V;

### T10I+D39R+R64S+E96K+N196W+S199G+A222V+E284Q;

### T10I+D39R+R64S+N196W+S199G+A222V+E284Q;

### T10I+R64S+E96K+N196W+S199G+A222V+E284Q;

### D39R+R64S+D90E+E96K+N196W+S199G+A222V+E284Q;

### T10I+R64S+E96K+N196W+S199G+A222V;

### T10I+D39R+N196W+S199G+A222V+E284Q;

### D39R+E96K+N196W+S199G+A222V+E284Q;

### D39R+R64S+N196W+S199G+A222V+E284Q;

### R64S+E96K+N196W+S199G+A222V+E284Q;

### D39R+R64S+N196W+S199G+A222V;

### S12*+S13*+V14*+K15*+N16*+1103Y+N196W+S199G+A222V+N233S+T308Y;

### S12*+S13*+V14*+K15*+N16*+A43D+1103Y+N196W+S199G+A222V+N233S+T308M;

### V9L+S12P+V14I+N16S+A43T+N196W+S199G+A222V;

### S12*+S13*+V14*+K15*+N16*+N196W+S199G+A222V;

### N28W+N196W+S199G+A222V;

### N196W+S199G+A222V+N392W+K417W;

### T10I+D39R+E96K+N196W+S199G+A222V;

### V9D+R38H+N196W+S199G+A222V+T348K;

### S113F+L150Y+N156K+N196W+S199G+A222V+H626*;

### S113Y+L150Y+N156K+N196W+S199G+A222V+H626*;

### S113W+L150Y+N156K+N196W+S199G+A222V+H626*;

### S113F+N156K+N196W+S199G+A222V+H626*;

### S113Y+N156K+N196W+S199G+A222V+H626*;

### S113W+N156K+N196W+S199G+A222V+H626*;

### W138Y+L150V+N196W+S199G+A222V+H626*;

### W138Y+L150V+D179G+N196W+S199G+A222V+H626*;

### W138Y+L150V+N196W+S199G+L218W+A222V+H626*;

### E96K+Q134L+D179S+N196W+S199G+A222V+E284Q+H626*;

### E96K+Q134L+L150Y+N156R+D179S+N196W+S199G+A222V+E284Q+H626*;

### E96K+L150Y+N156R+D179S+N196W+S199G+A222V+E284Q+H626*;

### R38H+E96K+G99N+K101R+Q134L+D179S+N196W+S199G+S221N+A222V+H626*;

### R38H+E96K+G99N+K101R+Q134L+D179S+N196W+S199G+A222V+H626*;

### R38H+E96K+G99N+K101R+Q134L+L150Y+N156R+D179S+N196W+S199G+A222V+H626*;

### R38H+E96K+G99N+K101R+L150Y+N156R+D179S+N196W+S199G+A222V+H626*; L150F+N196W+S199G+A222I+H626*;

### Q134L+L150F+N156R+N196W+S199G+A222I+H626*;

### Q134L+L150H+N156R+N196W+S199G+A222I+H626*;

### Q134L+L150M+N156K+N196W+S199G+A222I+H626*;

### Q134L+L150Y+N156K+N196W+S199G+A222I+Q457L+H626*;

### Q134M+L150W+N156K+N196W+S199G+A222I+H626*;

### Q134W+L150W+N156K+N196W+S199G+A222I+H626*;

### L150W+L152M+N156K+N196W+S199G+A222I+H626*;

### S113F+L150W+N156K+N196W+S199G+A222I+H626*;

### S113Y+L150W+N156K+N196W+S199G+A222I+H626*;

### L150W+G151W+N156K+N196W+S199G+A222I+H626*;

### L150W+G151S+N156K+N196W+S199G+A222I+H626*;

### S113F+L150W+G151S+N156K+N196W+S199G+A222I+H626*;

### Y67W+W68Y+L150W+N156K+N196W+S199G+A222I+H626*;

### A43V+L150M+G151F+N156R+N196W+S199G+A222I;

### L150M+G151Y+N156R+N196W+S199G+A222I;

### L150M+G151W+N156R+N196W+S199G+A222I;

### L150M+G151S+N156R+N196W+S199G+A222I+Y534H;

### S113F+L150M+G151S+N156R+N196W+S199G+A222I;

### Q134L+L150F+N156K+N196W+S199G+A222I+H626*;

### L150W+G151F+N156K+N196W+S199G+A222I+H626*;

### L150W+G151Y+N156K+N196W+S199G+A222I+H626*;

### Y67W+W68Y+L150W+N156K+N196W+S199G+A222I;

### G56W+N57P+Y67W+W68Y+L150W+N156K+N196W+S199G+A222I+H626*;

### K54I+Y67W+W68S+S113G+N196W+S199G+A222V+A382T+H626*;

### K54I+Y67W+W68S+S113G+D114Q+L150V+N196W+S199G+A222V+H626*;

### K54I+Y67W+W68S+S113G+W138Y+N196W+S199G+A222V+H626*;

### K54I+Y67W+W68S+S113G+D114Q+W138Y+L150V+N196W+S199G+A222V+H626*;

### Q134L+N196W+S199G+A222V+H626*;

### V107T+I108L+H110D+F169H+N196W+S199G+A222V+H626*;

### N196W+S199G+A222V+N392W+K417W+H626*;

### V9D+R38H+N196W+S199G+A222V+T348K+H626*;

### E96H+L150Y+N156R+N196W+S199G+A222V+E284Q+H626*;

### Q134L+L150Y+N196W+S199G+A222V+H626*;

### L150Y+N196W+S199G+A222V+H626*;

### Q134L+L150Y+N196W+S199G+A222V+H626*;

### L150F+N156R+N196W+S199G+A222V+H626*;

### L150H+N156R+N196W+S199G+A222V+H626*;

### Q134L+L150F+N156R+N196W+S199G+A222V+H626*;

### Q134L+L150H+N156R+N196W+S199G+A222V+H626*;

### Q134L+L150F+N156K+N196W+S199G+A222V+H626*;

### Q134L+L150H+N156K+N196W+S199G+A222V+H626*;

### Q134L+L150Y+N156K+N196W+S199G+A222V+H626*;

### L150Y+N156K+N196W+S199G+A222V+H626*;

### Q134L+L150Y+N156K+N196W+S199G+A222V+H626*;

### Q134W+L150Y+N156K+N196W+S199G+A222V+H626*;

### Q134M+L150Y+N156K+N196W+S199G+A222V+H626*;

### Q134M+L150Y+N156K+N196W+S199G+A222V+A466V+H626*;

### L150Y+L152M+N156K+N196W+S199G+A222V+H626*;

### L150S+N196W+S199G+A222V+H626*;

### N196W+S199G+A222V+N281S+H626*;

### Y67T+N196W+S199G+A222V+H626*;

### Q71N+N196W+S199G+A222V+H626*;

### Q71N+A94D+N196W+S199G+A222V+H626*;

### N196W+S199G+L218F+A222V+H626*;

### N196W+S199G+L218W+A222V+H626*;

### N196W+S199G+A222V+T278W+H626*;

### N196W+S199G+A222V+T278W+T459M+H626*;

### N196W+S199G+A222V+T278Y+H626*;

### N196W+S199G+A222V+S275N+H626*;

### N196W+S199G+A222V+S275L+H626*;

### N196W+S199G+A222V+S335Q+H626*;

### N196W+S199G+A222V+S335K+H626*;

### N196W+S199G+A222V+S335R+H626*;

### N196W+S199G+L218W+A222V+S335K+H626*;

### N196W+S199G+L218W+A222V+S335Q+H626*;

### Y67W+N196W+S199G+A222V+H626*;

### N196W+S199G+A222V+N281Q+H626*;

### L150M+N156R+N196W+S199G+A222V+H626*;

### Q134L+L150M+N156R+N196W+S199G+A222V+H626*;

### Q134L+L150M+N156K+N196W+S199G+A222V+H626*;

### D39R+N196W+S199G+A222V+N281Q+E284Q;

### D39R+N196W+S199G+A222V+E284Q+Q479QP;

### D39R+Y70F+N196W+S199G+A222V+E284Q;

### N28W+D39R+N196W+S199G+A222V+E284Q;

### D39R+N196W+S199G+N207W+A222V+E284Q;

### E1*+T2*+A3*+N4*+K5*+S6*+N7*+K8*+D39R+N196W+S199G+N207W+A222V+E284Q+H626*;

### N196W+N207W;

### N196W+N207W+E284Q;

### E1*+T2*+A3*+N4*+K5*+S6*+N7*+K8*+N196W+N207W+E284Q;

### L150Y+N196W+S199G+A222V+H626*;

### E1*+T2*+A3*+N4*+K5*+S6*+N7*+K8*+L150Y+N196W+S199G+A222V;

### E96K+K101R+L150M+N156R+D179S+N196W+S199G+N207W+T208N+A222V;

### E1*+T2*+A3*+N4*+K5*+S6*+N7*+K8*+E96K+K101R+L150M+N156R+D179S+N196W+S199G+T208N+A222V;

### Q134L+L150H+N156R+N196W+S199G+A222I+E284Q+H626*;

### E1*+T2*+A3*+N4*+K5*+S6*+N7*+K8*+Q134L+L150H+N156R+N196W+S199G+A222I+H626*;

### E1*+T2*+A3*+N4*+K5*+S6*+N7*+K8*+Q134L+L150H+N156R+N196W+S199G+N207W+A222I+E284Q;

### E96K+D179S+N196W+S199G+A222V+E284Q+H626*;

### E1*+T2*+A3*+N4*+K5*+S6*+N7*+K8*+E96K+D179S+N196W+S199G+A222V+E284Q;

### R64S+E96K+N196W+S199G+N207W+A222V;

### N28W+D39R+N196W+S199G+N207W+A222V+E284Q;

### D39R+N196W+S199G+A222V+E284Q+H626*;

### D39R+N196W+S199G+N207W+A222V+E284Q;

### E1*+T2*+A3*+N4*+K5*+S6*+N7*+K8*+D39R+N196W+S199G+A222V+E284Q;

### E1*+T2*+A3*+N4*+K5*+S6*+N7*+K8*+N196W+N207W+E511D;

### L150Y+N196W+S199G+N207W+A222V;

### E96K+K101R+L150M+N156R+D179S+N196W+S199G+T208N+A222V+H626*;

### E96K+K101R+L150M+N156R+D179S+N196W+S199G+T208N+A222V+E284Q;

### E1*+T2*+A3*+N4*+K5*+S6*+N7*+K8*+E96K+K101R+L150M+N156R+D179S+N196W+S199G+N207W+T208N+A222V+E284Q+H626*;

### Q134L+L150H+N156R+N196W+S199G+A222I;

### N28W+E96K+D179S+N196W+S199G+A222V+E284Q;

### R64S+E96K+N196W+S199G+A222V+H626*;

### R64S+E96K+N196W+S199G+A222V+E284Q;

### E1*+T2*+A3*+N4*+K5*+S6*+N7*+K8*+R64S+E96K+N196W+S199G+A222V;

and wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

The variants of the present disclosure to be applied in the process of the invention may in a particular embodiment, further comprise a substitution corresponding to K8N. More particularly when expressing the variants in a yeast host cell.

The variant polypeptide to be applied in the process of the invention may be a hybrid polypeptide in which a region of one polypeptide is fused at the N-terminus or the C-terminus of a region of another polypeptide.

E.g., in one embodiment the catalytic domain of the variant alpha-amylases may be fused to a Carbohydrate Binding Module (CBM) from another enzyme thus forming a hybrid alpha-amylase, wherein the catalytic core and the CBM are heterologous meaning that they do not occur in nature and that the CBM is foreign or heterologous to the catalytic domain.

Therefore, in a further embodiment a variant catalytic domain fragment, comprising a catalytic domain corresponding to at least amino acids amino acids 12-438 of SEQ ID NO: 1, preferably amino acids 1-438 of SEQ ID NO: 1, wherein optionally a linker and/or a carbohydrate binding module, CBM, has been replace with a heterologous CBM, may be applied in a process of the invention.

Heterologous meaning that the catalytic core and the CBM is not naturally occurring in the same polypeptide or enzyme.

In one embodiment, the CBM comprises amino acids 527-626 of SEQ ID NO: 1, and amino acids 439-526 comprises a linker region.

In a further embodiment, the heterologous CBM is selected from a CBM belonging to Family 20, 21, 25, 26, 34, 41 or 48. Preferably, the CBM is a Family 20 CBM.

In one particular embodiment the CBM is selected from the group consisting of:

i) a polypeptide of SEQ ID NO: 6, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 6;
ii) a polypeptide of SEQ ID NO: 7, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 7;
iii) a polypeptide of SEQ ID NO: 8, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 8;
iv) a polypeptide of SEQ ID NO: 9, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 9;
v) a polypeptide of SEQ ID NO: 10, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 10; and
vi) a polypeptide of SEQ ID NO: 11, or a polypeptide having at least 65%, at least 70%, at least 75%, at least 80%, at least 85%, at least 90%, at least 95%, at least 96%, at least 97%, at least 98%, or at least 99% sequence identity to the polypeptide of SEQ ID NO: 11.

It is well known in the art that linker regions may differ in length among different alpha-amylases and glucoamylases, and it is common that the length may vary in the range from about 1 amino acid to about 100 amino acids. Thus, in one embodiment the linker is selected to be in the range from 1-100 amino acids.

The variants applied in the process of the invention have been selected based on improved pH 4 stability. More particularly the variants have increased pH stability at pH 4.0, 32° C.-37° C., compared to a parent alpha-amylase particularly the alpha-amylase disclosed as SEQ ID NO: 1.

In one embodiment the variant alpha-amylases have increased pH stability at pH 4.0 compared to a parent alpha-amylase, and wherein increased pH stability at pH 4.0 can be determined as % residual alpha-amylase activity (% RA) after incubation of the variant amylase at pH 4.0, 32° C., for 18-24 hours. The Residual activity (RA %) after stress was calculated by dividing activity of stressed samples with unstressed samples and multiplying with 100. Alpha-amylase activity may e.g., be determined using the pNP-G7 alpha-amylase activity assay as described in the examples and in the material and methods section.

The improved stability may also be calculated as enzyme half-life in hours.

In a particular aspect the half-life is increased compared to the amylase of SEQ ID NO: 1 of at least a factor 1.2, 1.3, 1.4, 1.5, 2.0, 2.5, 3.0, 3.5, 4.0, 4.5, 5.0, 6.0, 7.0, such as at least 8.0.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Parent Alpha-Amylases

The parent alpha-amylase may be a polypeptide having at least 60% sequence identity to the polypeptide of SEQ ID NO: 1.

In an aspect, the parent has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have alpha-amylase activity. In one aspect, the amino acid sequence of the parent differs by up to 10 amino acids, e.g., 1, 2, 3, 4, 5, 6, 7, 8, 9, or 10, from the polypeptide of SEQ ID NO: 1. In another aspect, the parent comprises or consists of the amino acid sequence of SEQ ID NO: 1. In another aspect, the parent is a fragment of the polypeptide of SEQ ID NO: 1 containing at least the catalytic domain.

The parent polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from bacteria of the genus Bacillus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis alpha-amylase.

In one particular aspect, the parent is a Bacillus licheniformis.

In another aspect, the parent is a Bacillus amyloliquefaciens alpha-amylase, e.g., the alpha-amylase of SEQ ID NO: 1.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

Variant alpha-amylase suitable for use in the process of the present invention may be obtained by methods known in the art.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Nucleic Acid Constructs

The present disclosure also relates to nucleic acid constructs comprising a polynucleotide encoding a variant alpha-amylase operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a non-translated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present disclosure also relates to recombinant expression vectors comprising a polynucleotide encoding a variant suitable for use in the process of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-am inoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant suitable for use in the process of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present disclosure also relates to methods of producing a variant suitable for use in the process of the invention, comprising (a) cultivating a recombinant host cell under conditions conducive for production of the variant; and optionally (b) recovering the variant.

The recombinant host cells are cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Fermentation Broth Formulations or Cell Compositions

The present disclosure also relates to a fermentation broth formulation or a cell composition comprising a variant suitable for use in the process of the present invention. The fermentation broth product further comprises additional ingredients used in the fermentation process, such as, for example, cells (including, the host cells containing the gene encoding the variant of the present invention which are used to produce the variant of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present disclosure also relates to compositions comprising a variant of the present invention. Preferably, the compositions are enriched in such a variant. The term “enriched” indicates that the alpha-amylase activity of the composition has been increased, e.g., with an enrichment factor of at least 1.1.

The compositions may comprise a variant of the present invention as the major enzymatic component, e.g., a mono-component composition. Alternatively, the compositions may comprise multiple enzymatic activities, such as one or more enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

In a particular embodiment, the composition comprises the variant alpha-amylase of the invention and a glucoamylase.

In another embodiment, the composition comprises the variant alpha-amylase of the invention and another alpha-amylase.

In another embodiment, the composition comprises the variant alpha-amylase of the invention, a glucoamylase, and another alpha-amylase.

In an embodiment, the glucoamylase comprised in the composition is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, preferable P. sanguineus, or a strain of Gloeophyllum, such as G. sepiarium or G. trabeum, or a strain of the Nigrofomes.

In an embodiment the glucoamylase is derived from Trametes, such as a strain of Trametes cingulata, such as the one shown in SEQ ID NO: 4 herein.

In an embodiment the glucoamylase is selected from the group consisting of:

(i) a glucoamylase comprising the polypeptide of SEQ ID NO: 4 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 4 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SANT™ SUPER, SANT™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

### Methods of Using the Alpha-Amylase of the Invention—Industrial Applications

The variant alpha-amylases of the present disclosure possess valuable properties allowing for a variety of industrial applications. In particular, the variant alpha-amylases may be used in ethanol production, and starch conversion processes.

Further, the alpha-amylases of the disclosure are particularly useful in the production of ethanol, such as fuel, drinking and industrial ethanol, from whole grains.

In one embodiment, the present invention relates to a use of the alpha-amylase according to the disclosure in a saccharification process, particularly a simultaneous saccharification and fermentation process.

Production of Fermentation Products

Fermentable sugars (e.g., dextrins, monosaccharides, particularly glucose) are produced from enzymatic saccharification. These fermentable sugars may be further purified and/or converted to useful sugar products. In addition, the sugars may be used as a fermentation feedstock in a microbial fermentation process for producing end-products, such as alcohol (e.g., ethanol, and butanol), organic acids (e.g., succinic acid, 3-HP and lactic acid), sugar alcohols (e.g., glycerol), ascorbic acid intermediates (e.g., gluconate, 2-keto-D-gluconate, 2,5-diketo-D-gluconate, and 2-keto-L-gulonic acid), amino acids (e.g., lysine), proteins (e.g., antibodies and fragment thereof).

In an embodiment, the fermentable sugars obtained during the liquefaction process steps are used to produce alcohol and particularly ethanol. In ethanol production, an SSF process is commonly used wherein the saccharifying enzymes and fermenting organisms (e.g., yeast) are added together and then carried out at a temperature of 30-40° C.

The organism used in fermentation will depend on the desired end-product. Typically, if ethanol is the desired end product yeast will be used as the fermenting organism. In some preferred embodiments, the ethanol-producing microorganism is a yeast and specifically Saccharomyces such as strains of S. cerevisiae (U.S. Pat. No. 4,316,956). A variety of S. cerevisiae are commercially available and these include but are not limited to FALI (Fleischmann's Yeast), SUPERSTART (Alltech), FERMIOL (DSM Specialties), RED STAR (Lesaffre) and Angel alcohol yeast (Angel Yeast Company, China). The amount of starter yeast employed in the methods is an amount effective to produce a commercially significant amount of ethanol in a suitable amount of time, (e.g., to produce at least 10% ethanol from a substrate having between 25-40% DS in less than 72 hours). Yeast cells are generally supplied in amounts of about 10⁴ to about 10¹², and preferably from about 10⁷ to about 10¹⁰ viable yeast count per mL of fermentation broth. After yeast is added to the mash, it is typically subjected to fermentation for about 24-96 hours, e.g., 35-60 hours. The temperature is between about 26-34° C., typically at about 32° C., and the pH is from pH 3-6, e.g., around pH 4-5.

The fermentation may include, in addition to a fermenting microorganisms (e.g., yeast), nutrients, and additional enzymes, including phytases. The use of yeast in fermentation is well known in the art.

Processes for Producing Fermentation Products from Un-Gelatinized Starch-Containing Material

The invention relates to processes for producing fermentation products from starch-containing material without gelatinization (i.e., without cooking) of the starch-containing material (often referred to as a “raw starch hydrolysis” process). The fermentation product, such as ethanol, can be produced without liquefying the aqueous slurry containing the starch-containing material and water. In one embodiment a process of the invention includes saccharifying (e.g., milled) starch-containing material, e.g., granular starch, below the initial gelatinization temperature, preferably below 57° C., preferably in the presence of an alpha-amylase of the disclosure and a glucoamylase to produce sugars that can be fermented into the fermentation product by a suitable fermenting organism. In this embodiment the desired fermentation product, e.g., ethanol, is produced from un-gelatinized (i.e., uncooked), preferably milled, cereal grains, such as corn.

Accordingly, in one aspect the invention relates to processes for producing a fermentation product from starch-containing material comprising simultaneously saccharifying and fermenting starch-containing material using a carbohydrate-source generating enzymes and a fermenting organism at a temperature below the initial gelatinization temperature of said starch-containing material in the presence of an alpha-amylase of the disclosure.

In particular, the resent invention relates to a process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out using at least a variant alpha-amylase comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

Preferably the starch-containing material is corn, the fermentation product is ethanol, and steps a) and b) are carried out simultaneously.

In one embodiment, the fermenting organism expresses the variant alpha-amylase of the disclosure and/or a glucoamylase.

In another preferred embodiment of the process, the fermenting organism is a yeast cell, particularly a Saccharomyces cell, such as Saccharomyces cerevisiae.

The fermentation product, e.g., ethanol, may optionally be recovered after fermentation, e.g., by distillation. Typically, amylase(s), such as glucoamylase(s) and/or other carbohydrate-source generating enzymes, and/or alpha-amylase(s), is(are) present during fermentation. Examples of glucoamylases and other carbohydrate-source generating enzymes include raw starch hydrolyzing glucoamylases. The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. Before initiating the process, a slurry of starch-containing material, such as granular starch, having 10-55 w/w % dry solids (DS), preferably 25-45 w/w % dry solids, more preferably 30-40 w/w % dry solids of starch-containing material may be prepared. The slurry may include water and/or process waters, such as stillage (backset), scrubber water, evaporator condensate or distillate, side-stripper water from distillation, or process water from other fermentation product plants. Because the process of the invention is carried out below the initial gelatinization temperature, and thus no significant viscosity increase takes place, high levels of stillage may be used if desired. In an embodiment the aqueous slurry contains from about 1 to about 70 vol. %, preferably 15-60 vol. %, especially from about 30 to 50 vol. % water and/or process waters, such as stillage (backset), scrubber water, evaporator condensate or distillate, side-stripper water from distillation, or process water from other fermentation product plants, or combinations thereof, or the like. The starch-containing material may be prepared by reducing the particle size, preferably by dry or wet milling, to 0.05 to 3.0 mm, preferably 0.1-0.5 mm. After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material are converted into a soluble starch hydrolyzate. A process in this aspect of the invention is conducted at a temperature below the initial gelatinization temperature, which means that the temperature typically lies in the range between 30-57° C. In a preferred embodiment the process carried at a temperature from 25° C. to 40° C., such as from 28° C. to 35° C., such as from 30° C. to 34° C., preferably around 32° C. In an embodiment the process is carried out so that the sugar level, such as glucose level, is kept at a low level, such as below 6 w/w %, such as below about 3 w/w %, such as below about 2 w/w %, such as below about 1 w/w %, such as below about 0.5 w/w %, or below 0.25 w/w %, such as below about 0.1 w/w %. Such low levels of sugar can be accomplished by simply employing adjusted quantities of enzyme and fermenting organism. A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 w/w %, such as below about 0.2 w/w %. The process of the invention may be carried out at a pH from about 3 and 7, preferably from pH 3.5 to 6, or more preferably from pH 4 to 5. In an embodiment fermentation is ongoing for 6 to 120 hours, in particular 24 to 96 hours.

Glucoamylase Present and/or Added in Saccharification and/or Fermentation

A glucoamylase is present and/or added in saccharification and/or fermentation, preferably simultaneous saccharification and fermentation (SSF), in a process of the invention (i.e., oil recovery process and fermentation product production process).

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SANT™ SUPER, SANT™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

Alpha-Amylase Present and/or Added in Saccharification and/or Fermentation

In an aspect a variant alpha-amylase of the disclosure is present and/or added in saccharification and/or fermentation in a process of the invention. In a preferred embodiment the alpha-amylase is of bacterial origin.

In a preferred embodiment the alpha-amylase present and/or added in saccharification and/or fermentation is derived from a strain of the genus Bacillus, preferably a strain the Bacillus licheniformis, such as one shown in SEQ ID NO: 1 herein.

In a preferred embodiment, the ratio between glucoamylase and variant alpha-amylase present and/or added during saccharification and/or fermentation may preferably be in the range from 500:1 to 1:1, such as from 250:1 to 1:1, such as from 100:1 to 1:1, such as from 100:2 to 100:50, such as from 100:3 to 100:70.

Fermentation Medium

The environment in which fermentation is carried out is often referred to as the “fermentation media” or “fermentation medium”. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

Starch-Containing Materials

Any suitable starch-containing material may be used according to the present invention. The starting material is generally selected based on the desired fermentation product. Examples of starch-containing materials, suitable for use in a process of the invention, include whole grains from corn,

Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol) In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. The fermentation product, such as ethanol, obtained according to the invention, may be used as fuel, which is typically blended with gasoline. However, in the case of ethanol it may also be used as potable ethanol.

Recovery of Fermentation Products

Subsequent to fermentation, or SSF, the fermentation product may be separated from the fermentation medium. The slurry may be distilled to extract the desired fermentation product (e.g., ethanol). Alternatively, the desired fermentation product may be extracted from the fermentation medium by micro or membrane filtration techniques. The fermentation product may also be recovered by stripping or other method well known in the art.

The present invention is further described by the following examples that should not be construed as limiting the scope of the invention.

### EXAMPLES

### Strains

Bacillus amyloliquefaciens strain used in the examples was isolated from soil in Virginia in the USA in 2011.

Saccharomyces cerevisiae strain MBG5012 (deposited under Accession No. NRRL Y67700 at the Agricultural Research Service Patent Culture Collection 5 (NRRL), USA, and described in WO19/161227.

### Materials and Methods

### Material

TB-GLY media

### MSA-SUB-FS-057

| Raw material | Amount | |
|---|---|---|
| Tryptone | 13.3 | g |
| Yeast extract | 26.6 | g |
| Glycerol anhydrous pure | 5.5 | g |
| Ion exchanged water up to | 1000 | ml |

The 100 mM BR buffer is prepared from a 1M BR stock by adding 50 μL of a 2M CaCl₂) solution, and 1000 μL of a 10% Brij35 stock solution to 100 mL 1M stock, and 900 mL milliQ water to a concentration of 0.1 mM CaCl₂) and 0.01% Brij35.

The 1M BR stock is prepared by dissolving 136 g Sodium acetate dihydrate (CAS 6131-90-4), 142 g Disodium hydrogen phosphate (CAS 7558-79-4), and 61.8 g Boric acid (CAS 10043-35-3) in milliQ water to a total volume of 1 L.

After dilution, the pH of the 100 mM BR buffer is adjusted to pH 4 or pH 7 by adding HCL or NaOH as needed.

### Assays:

pNP-G7 Alpha-Amylase Activity Assay

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolyzed substrate further to liberate a free PNP molecule which has a yellow color and thus can be measured by visible spectrophotometry at λ=405 nm (400-420 nm.). Kits containing G7-pNP substrate and alpha-Glucosidase is manufactured by Roche/Hitachi (cat. No. 11876473) or Sigma-Aldrich (Catalog number MAK009).

### Reagents:

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≥4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O (C₂H₄O), (n=9-10))), 1 mM CaCl₂), pH8.0.

### Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Amylase pH 4 Stability Screening Assay

### Sample Preparation

The expression clones are grown between 18 hours and 24 hours under good expression conditions in TB-GLY medium in 2.2 mL deep-well plates at 37° C. at 700 RPM in a TH 15 TiMix microplate shaker from Edmund Buhler Gmbh.

After fermentation, the samples can be spun down to reduce the number of cells in the supernatant.

### Sample Incubation

From each variant supernatant sample, a subsample was incubated at pH 4 and 37° C. for 18 to 24 hours in a 100 mM BR-buffer adjusted to pH 4 (hereafter called the stressed sample), while an equivalent subsample was stored at pH 7 at 4° C. or below in a 100 mM BR buffer adjusted to pH 7 (hereafter called the unstressed sample). Incubation was performed in a 96-well Nunc MicroWell polypropylene plate (Sigma Aldrich P6866) sealed with an Agilent PlateLoc plate-sealer.

10 μL of variant supernatant sample was diluted 10 times by mixing it into 90 μl buffer for incubation.

### Sample Analysis

After incubation, the samples were prediluted 20 before activity was measured. Amylase activity was determined using a G7-pNP kit (Sigma-Aldrich Catalog number MAK009). Sample dilution was performed in the pH 7 BR buffer. A further dilution was done to a total in-assay dilution of 1000×, by mixing 10 μL of diluted sample with 40 μL G7 substrate in a 380-well PerkinElmer SpectraPlate Reader-Plate.

The stressed and unstressed sample of a given variant were read on the same reader-plate. The enzyme activity was evaluated according to the G7 assay protocol. The solution was mixed and pre-incubated 1 minute at room temperature and absorption was measured every 20 sec. over 30 minutes at OD 405 nm, and the highest measured slope was used as a measure of enzyme activity. A well with no enzyme sample added was used as control, and any slope in that well was subtracted from all the measured activities on the same plate.

### Data Evaluation—Determination of Half-Life

For a given variant, the enzyme activity of the stressed sample was divided by the enzyme activity of the unstressed sample, to compute residual activity. From this, the half-life in hours of the enzyme candidate is computed as the negative of the incubation-time in hours divided by log 2 of the residual activity.

### Example 1: Cloning and Expression of the Parent Wild Type Bacillus amyloliquefaciens Alpha-Amylase and Variants Thereof

The strain Bacillus amyloliquefaciens was isolated from soil in Virginia in the USA in 2011. Chromosomal DNA from the strain was subjected to full genome sequencing using Illumina technology. The GH13 subfamily 28 amylase was identified in the genome by analysing for glycosyl hydrolase domains (according to the CAZY definition).

A linear integration vector-system was used for the expression cloning of the amylase from Bacillus amyloliquefaciens. The linear integration construct was a PCR fusion product made by fusion of the gene between two Bacillus subtilis homologous chromosomal regions along with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003/095658. The gene was expressed under the control of a triple promoter system (as described in WO 1999/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene coding for chloramphenicol acetyl-transferase was used as marker (described in e.g. Diderichsen, B.; Poulsen, G. B.; Joergensen, S. T. 1993, Plasmid, “A useful cloning vector for Bacillus subtilis” 30:312. The final gene construct was integrated in the Bacillus chromosome by homologous recombination into the pectate lyase locus.

The gene encoding the amylase was amplified from chromosomal DNA of the strains with gene specific primers containing overhang to the two flanking vector fragments. The amylase was expressed with a Bacillus clausii secretion signal (amino acids 1-27 of SEQ ID NO: 3) replacing the genes native secretion signal and with a histidine tag of 6 histidines fused directly to the C-terminal of the protein. The polynucleotide sequence used for expression was included herein as SEQ ID NO: 2. The upstream and downstream vector fragments were amplified from genomic DNA of the strain MB1361 (based on strain PL3598 described in patent application WO 2003095658). The 2 linear vector fragments and the gene fragment were assembled into one linear vector construct by SOE PCR. An aliquot of the PCR product was transformed into Bacillus subtilis. Transformants were selected on LB plates supplemented with 6 μg of chloramphenicol per ml. A recombinant Bacillus subtilis clone containing the sequence confirmed integrated expression construct was cultivated in liquid culture on a rotary shaking table in 500 mL baffled Erlenmeyer flasks each containing 100 ml yeast extract-based media. The clone was cultivated for 4 days at 30° C. The enzyme containing supernatant was harvested and the enzyme purified as described in Example 2.

### Example 2: Purification of the His-Tagged Amylase from Bacillus amyloliquefaciens

The pH of the supernatant was adjusted to pH 8 with 3 M Tris, left for 1 hour, and then filtered using a filtration unit equipped with a 0.2 μm filter (Nalgene). The filtered supernatant was applied to a 5 ml HisTrap™ Excel column (GE Healthcare Life Sciences) pre-equilibrated with 5 column volumes (CV) of 50 mM Tris/HCl pH 8. Unbound protein was eluted by washing the column with 8 CV of 50 mM Tris/HCl pH 8. The amylase was eluted with 50 mM HEPES pH 7-10 mM imidazole and elution was monitored by absorbance at 280 nm. The eluted amylase was desalted on a HiPrep™ 26/10 desalting column (GE Healthcare Life Sciences) pre-equilibrated with 3 CV of 50 mM HEPES pH 7-100 mM NaCl. The amylase was eluted from the column using the same buffer at a flow rate of 10 ml/minute. Relevant fractions were selected and pooled based on the chromatogram and SDS-PAGE analysis using 4-12% Bis-Tris gels (Invitrogen) and 2-(N-morpholino)ethanesulfonic acid (MES) SDS-PAGE running buffer (Invitrogen). The gel was stained with InstantBlue (Novexin) and destained using miliQ water. The concentration of the purified enzyme was determined by absorbance at 280 nm.

### Example 3: Determination of pH Stability for Variant Alpha-Amylases

The pH stability of the amylase variants according to the invention were tested as described above using the G7-pNP kit (Sigma-Aldrich Catalog number MAK009). After growing the variants as described, supernatant samples of each variant were tested for increased stability at pH 4 over the parent amylase. A subsample was incubated at pH 4 and 37° C. for 19.5 or 21 hours in a 100 mM BR-buffer adjusted to pH 4 (hereafter called the stressed sample), while an equivalent 5 subsample was stored at pH 7 at 4° C. or below in a 100 mM BR buffer adjusted to pH 7 (hereafter called the unstressed sample). Incubation was performed in a 96-well Nunc MicroWell polypropylene plate (Sigma Aldrich P6866) sealed with an Agilent PlateLoc plate-sealer.

After incubation, the samples were prediluted 20 to 200 times before activity was measured. Amylase activity was determined using a G7-pNP kit (Sigma-Aldrich Catalog number MAK009). Sample dilution was performed in the pH 7 BR buffer. A further dilution was done to a total in-assay dilution of 100× to 1000×. 10 μL of diluted sample is mixed with 40 μL G7 substrate in a 380-well PerkinElmer SpectraPlate reader-plate.

The stressed and unstressed sample of each variant were read on the same reader-plate. The enzyme activity was evaluated according to the G7 assay protocol. The solution was mixed and pre-incubated 1 minute at room temperature and absorption was measured every 20 sec. over 30 minutes at OD 405 nm, and the highest measured slope was used as a measure of enzyme activity. A well with no enzyme sample added was used as control, and any slope in that well was subtracted from all the measured activities on the same plate.

The results of the stability test are shown in Table 1 and 2 below.

TABLE 1
Half-life for variants stressed at pH 4.0, 37° C. for 19.5 hours

| Variant substitutions | IF | HIF | Half Life (hours) |
|---|---|---|---|
| A222I | 8.1 | 4.0 | 19.4 |
| A222V | 7.9 | 3.9 | 18.7 |
| S199G | 7.8 | 3.8 | 18.6 |
| N196W, N207W, N603W | 7.2 | 3.4 | 16.7 |
| N196W, N603W | 7.1 | 3.4 | 16.5 |
| N196W, N207W | 6.8 | 3.2 | 15.6 |
| A222E | 4.8 | 2.3 | 11.1 |
| N207W, N603W | 1.4 | 1.1 | 5.5 |
| N603W | 1.3 | 1.1 | 5.4 |
| Wt parent amylase | 1.0 | 1.0 | 4.8 |
| SEQ ID NO: 1 | | | |

TABLE 2
Half-life for variants stressed at pH 4.0, 37° C. for 21 hours

| Variant substitution | HIF | Half Life (hours) |
|---|---|---|
| S199G | 3.94 | 16.82 |
| A222V | 3.86 | 16.46 |
| A222I | 3.85 | 16.42 |
| N196W | 3.48 | 14.84 |
| A222E | 2.90 | 12.38 |
| Wt parent amylase | 1.08 | 4.61 |
| SEQ ID NO: 1 | | |

The results show that all tested variants had increased stability at pH 4.0 compared to the wild type parent alpha-amylase of SEQ ID NO: 1.

### Example 4: Determination of pH 4 Stability for Variants Alpha-Amylases in Presence of Raw Starch

Amylase variants were tested as supernatants from fermentation and purified samples. Purified enzyme samples were diluted in water with 0.01% Brij to a concentration of 5 μM. For the assay, 10 μl of supernatant or diluted purified sample was mixed with 90 μl 10% raw corn starch in 100 mM BR buffer, pH 4. Each sample was prepared on two separate plates, where one was stressed by incubation for 18 hours at 37° C., 850 rpm (hereafter called stressed samples) and the second was incubated for 2 minutes at room temperature, 850 rpm (hereafter called unstressed samples). After respective incubation, both plates were spun down for 2 min at 2000 rpm and supernatants were stored at −20° C. until analysis. Defrosted supernatants were diluted 10× in 100 mM BR buffer, pH 7 and enzyme activity was evaluated using G7-pNP assay protocol (Roche/Hitachi, cat. no. 11876473). Briefly, 20 μl diluted enzyme sample was mixed with 100 μl G7-pNP solution and absorbance was followed at 405 nm for 20 min at room temperature. Initial slopes (0-2 min), after blank subtraction, were used as activity measure. Residual activity after stress was calculated by dividing activity of stressed samples with unstressed samples. From this, the half-life (T½) in hours of the enzyme candidate was computed as the incubation-time in hours divided by log 2 of the residual activity multiplied by −1.

Improvement factor (IF) was calculated from the estimated half-life (T½), by dividing the estimated T½ for variants with the T½ of the wild type enzyme (SEQ ID NO:1).

TABLE 3
Half-life for variants stressed at pH 4.0, 37°
C. for 18 hours in presence of raw starch

| | Supernatant samples | | | Purified samples | | |
|---|---|---|---|---|---|---|
| | RA @ | T½ | IF | | RA @ | T½ |
| | 18 h | (hour) | (T½) | | 18 h | (hour) |
| Wt amylase | 7% | 4.9 | 1.0 | Ref. | 0% | |
| SEQ ID NO: 1 | | | | | | |
| N196W | 28% | 9.8 | 2.0 | N196W | 23% | 8.7 |

The results show that listed variants had increased stability at pH 4.0 compared to the wild type parent alpha-amylase of SEQ ID NO: 1.

### Example 5: Impact of Protein Engineering on B. amyloliquefaciens Alpha-Amylase (SEQ ID NO: 1) to Improve Ethanol Yield and Increase Kinetics During Raw-Starch Corn Mash Fermentation

This example describes the evaluation of the alpha-amylase of SEQ ID NO: 1 containing a mutation for improved ethanol and kinetics during raw-starch corn mash simultaneous saccharification and fermentation (SSF). Particularly, the ethanol and kinetics during fermentation are compared among the enzymes listed in Table 4.

TABLE 4
List of enzymes used
Enzymes
Wt AA B. amyloliquefaciens (SEQ ID NO: 1)
N196W variant AA

### Seed Culture:

Cryo-preserved culture of Saccharomyces cerevisiae strain MBG5012 (deposited under Accession No. NRRL Y67700 at the Agricultural Research Service Patent Culture Collection 5 (NRRL), USA, and described in WO19/161227 (incorporated herein by reference in its entirety) was first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water and glycerol. The resuspended cells were used to inoculate the corn mash at the beginning of simultaneous saccharification and fermentation (SSF).

### Corn Mash:

Corn kernels were ground using the Turkish grind setting on a Bunn Coffee Grinder. The % dried solids (DS) of the corn flour was 84.50%. Using the Turkish ground corn and tap water, a slurry targeting 37.50% DS was prepared. The corn slurry was supplemented with 1000 ppm urea and 3 ppm of antibiotic LACTROL™ and its pH was adjusted to 4.5 prior to use in SSF. Final dried solids level was determined to be 37.50% DS.

### Simultaneous Saccharification and Fermentation (SSF)

All fermentations were carried out in 12 mL round-bottom tubes with caps having a drilled hole. Tubes were filled with 3.8-4.5 g of corn slurry and inoculated with seed culture at 10 million cells per gram mash. A glucoamylase from Trametes cingulate (disclosed herein as SEQ ID NO: 4) was added to the tubes at 88 μg enzyme protein per g of dry corn solids. Amylases in Table 4 were added to the tubes at 32 μg enzyme protein per g of dry corn solids. All tubes contained the same glucoamylase and one amylase from Table 4 was added per tube. Tubes were incubated in an incubator at 32° C. Tubes were vortexed two times per day. Fermentation was run for 72 hours.

### Weight Loss Analysis

Initial weight of tubes prior to fermentation was recorded. Weight of tubes was recorded after 18, 24, 48 and 72 h of fermentation. The difference in weight between 0 h and 18, 24, 48 and 72 h of fermentation was calculated. Weight difference was divided by g DS per tube resulting in g weight loss/g DS.

### Ethanol Analysis

After 72 hours of fermentation, 100 μl of 40% v/v H₂SO₄ was added to each sample tube, samples were vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

Table 5 and Table 6 show the CO₂ g weight loss kinetics/gDS and ethanol titers, respectively, for the amylases during corn mash fermentation. Results indicate that the fermentation kinetics and ethanol titer increase when the fermentation is run with a variant alpha-amylase comprising a N196W substitution versus the parent wt amylase. The difference in kinetics and ethanol titer can be attributed to the mutation present in the variant amylase. It can be concluded that the N196W substitution present in the variant provides improvement in fermentation performance by enabling increased activity on the starch substrate present in the raw-starch corn slurry.

TABLE 5
Gram weight loss/g DS kinetics
during raw-starch fermentation run with

| | 18 h g | 24 h g | 48 h g | 72 h g |
|---|---|---|---|---|
| | Weight | Weight | Weight | Weight |
| | Loss/g DS | Loss/g DS | Loss/g DS | Loss/g DS |
| Amylase | Mean | Mean | Mean | Mean |
| SEQ ID NO: 1 | 0.159 | 0.215 | 0.353 | 0.460 |
| N196W | 0.173 | 0.231 | 0.379 | 0.468 |

TABLE 6
Ethanol titer after 72 h of fermentation in
raw-starch slurry with amylases from Table 4

| | Mean Ethanol | Std |
|---|---|---|
| Amylase | %(v/v) | Dev |
| SEQ ID NO: 1 | 18.32 | 0.13 |
| N196W | 19.01 | 0.18 |

### Example 6. Determination of pH 4 Stability for Variant Alpha-Amylases in Presence and Absence of Raw Starch

Purified amylase variants were diluted in water with 0.01% Brij to a concentration of 5 μM. For the assay, 10 μl diluted purified sample was mixed with 90 μl 100 mM BR buffer, pH or with 10% raw corn starch in 100 mM BR buffer, pH 4. Each sample was prepared on two separate plates, where one was stressed by incubation for 96 hours at 32° C., 850 rpm (hereafter called stressed samples) and the second was incubated for 2 minutes at room temperature, 850 rpm (hereafter called unstressed samples). After respective incubation, both plates were spun down for 2 min at 2000 rpm and supernatants were stored at −20° C. until analysis. Defrosted supernatants were diluted 10× in 100 mM BR buffer, pH 7 and enzyme activity was evaluated using G7-pNP assay protocol (Roche/Hitachi, cat. no. 11876473). Briefly, 20 μl diluted enzyme sample was mixed with 100 μl G7-pNP solution and absorbance was followed at 405 nm for 20 min at room temperature. Initial slopes (0-2 min), after blank subtraction, were used as activity measure. Residual activity after stress was calculated by dividing activity of stressed samples with unstressed samples.

TABLE 7
Residual activities for variants stressed at pH 4.0, 32° C.
for 96 hours in absence or presence of raw starch.

| | Residual Activity | |
|---|---|---|
| Mutations relative | after incubation | Residual Activity |
| to WT parent amylase | in buffer pH 4, | after incubation |
| SEQ ID NO: 1 | with 10% starch | in buffer pH 4 |
| S199G H626* | 9 | 0 |
| L150H S199G A222I | >95 | 83 |
| L150M S199G A222V | 86 | 88 |
| N196W S199G A222V N603W | 82 | 91 |
| L150Y N196W S199G A222V | >95 | 93 |
| L150F N196W S199G A222I | >95 | >95 |
| L150M N196W S199G A222V | >95 | >95 |
| L150W S199G A222V | >95 | >95 |
| L150H N196W S199G A222V | >95 | >95 |
| N196W S199G A222V H626* | >95 | >95 |
| L150W N196W S199G A222I | >95 | >95 |
| N196W S199G A222V | >95 | >95 |
| L150Y N196W S199G A222V | >95 | >95 |
| L150Y S199G A222V | >95 | >95 |

The results show that listed variants had increased stability at pH 4.0 compared to the wild type parent alpha-amylase of SEQ ID NO: 1.

### Example 7. Determination of pH 4 Stability for Variant Alpha-Amylases

Purified amylase variants were diluted in water with 0.01% Trition-X-100 to a concentration of 5 μM. For the assay, the 5 μM samples were diluted 10× into either stress buffer (222 mM sodium acetate buffer pH 4, 0.56 mM CaCl₂) and 0.01% Brij-35) or dilution buffer (0.01% Trition-X-100). Samples in the stress buffer were incubated at 32° C. (850 rpm) for 24H (hereafter called stressed samples). The other samples mixed with dilution buffer were stored at 5° C. until further analysis, max. 2 days (hereafter called unstressed samples).

After the respective incubations, samples were diluted 10-50× with assay buffer (500 mM Hepes pH 7, 0.5 mM CaCl₂), 0.01% Brij-35) and enzyme activity was evaluated using G7-pNP assay protocol (Roche/Hitachi, cat. no. 11876473). Briefly, 20 μl diluted enzyme sample was mixed with 100 μl G7-pNP solution and absorbance was measured at 405 nm for 20 min at room temperature. Initial slopes (lag time: 2 min, max absorbance=2) were calculated and blank subtracted. These slopes—blank were used as activity measure. Residual activity (RA %) after stress was calculated by dividing activity of stressed samples with unstressed samples and multiplying with 100.

TABLE 8
Residual activities (RA %) for variants
stressed at pH 4.0, 32° C. for 24 hours

| | % RA after |
|---|---|
| Variant substitutions | 24 hrs at pH 4 |
| Wt parent amylase SEQ ID # 1 | 0 |
| A222V, H626* | 13.8 |
| A222I, H626* | 14.4 |
| A222E, H626* | 4.2 |
| N196W, H626* | 10.2 |
| L150H, H626* | 1.6 |
| L150M, H626* | 0.70 |
| L150Y, H626* | 6.3 |
| L150W | 7.9 |
| S199G | 18.1 |
| N196W, N603W, H626* | 8.3 |
| N196W, N207W, H626* | 11.3 |
| N196W, N207W, N603W, H626* | 9.4 |
| N196W, S199G, A222V | 65.1 |
| N196W, S199G, A222V, H626* | 64.0 |
| N196W, S199G, A222V, N603W | 66.6 |
| L150F, N196W, S199G, A222I | 94.7 |
| L150H, S199G, A222I | 55.6 |
| L150M, S199G, A222V | 65.5 |
| L150M, N196W, S199G, A222V | 64.2 |
| L150W, S199G, A222V | 61.7 |
| L150W, N196W, S199G, A222I | 90.3 |
| L150Y, S199G, A222V | 58.2 |
| L150Y, N196W, S199G, A222V | 80.9 |
| L150H, N196W, S199G, A222V | 71.0 |
| E96K, D179S, N196W, S199G, A222V, E284Q | 77.9 |
| R64S, E96K, N196W, S199G, A222V | 86.6 |

The results show that the listed variants had increased stability at pH 4.0 compared to the wild type parent alpha-amylase of SEQ ID NO: 1.

### Example 8. Determination of pH 4 Stability for Variant Alpha-Amylases

Purified amylase variants were diluted in water with 0.01% Trition-X-100 to a concentration of 5 μM. For the assay, the 5 μM samples were diluted 10× into either stress buffer (222 mM sodium acetate buffer pH 4, 0.56 mM CaCl₂) and 0.01% Brij-35) or dilution buffer (0.01% Trition-X-100). Samples in the stress buffer were incubated at 32° C. (850 rpm) for 24H (hereafter called stressed samples). The other samples mixed with dilution buffer were stored at 5° C. until further analysis (hereafter called unstressed samples).

After incubation, the stressed samples were diluted 10-50× with assay buffer (500 mM Hepes pH 7, 0.5 mM CaCl₂), 0.01% Brij-35). The unstressed samples were diluted between 10-100× to get minimum four different dilutions and thus minimum 4 different concentrations. Enzyme activity was evaluated for all diluted samples (stressed and unstressed) using G7-pNP assay protocol (Roche/Hitachi, cat. no. 11876473). Briefly, 20 μl diluted enzyme sample was mixed with 100 μl G7-pNP solution and absorbance was measured at 405 nm for 20 min at room temperature. Initial slopes (lag time: 2 min, max absorbance=1.5) were calculated. For the unstressed samples, a nonlinear fit (e.g. Michaelis Menten) was made using initial slopes as Y and enzyme concentration as X. Based on this fit, the concentration of residual active enzyme was estimated in the stressed samples. The level of residual activity (RA %) was calculated by dividing the estimated concentration of residual active enzyme with the initial concentration of enzyme (at start) and multiplying with 100.

The results are given in Table 9 below. The listed alterations were introduced into the parent alpha-amylase of SEQ ID NO: 1, and the results show that the listed variants had increased stability at pH 4.0 compared to the wild type parent alpha-amylase of SEQ ID NO: 1.

TABLE 9
Residual activities (RA%) for variants stressed at pH 4.0, 32° C. for 24 hours.

| | % RA after |
|---|---|
| Variant substitutions | 24 hrs at pH 42 |
| Wt parent amylase of SEQ ID NO: 1 | 0 |
| N28W, H626* | 12.8 |
| S199G, H626* | 21.6 |
| N196W, V599W, H626* | 11 |
| N196W, H550Y, P605S, H626* | 12.5 |
| N196W, A545P, T576Y, H626* | 8 |
| N196W, K549Y, G560P, H626* | 8.7 |
| I108P, Y183I, N196W, I205Y, H626* | 16.9 |
| N196W, R323K, H626* | 9.6 |
| N196W, D283P, H626* | 11.4 |
| W138Y, N196W, H626* | 9.6 |
| L150W, H626* | 7.2 |
| N196W, N392W, K417W, H626* | 8.3 |
| N196W, N392R, K417W, H626* | 15.6 |
| N196W, K549*, H550*, D551*, H626* | 14.6 |
| N196W, P580*, E581*, N582*, H626* | 14 |
| N196W, F592FK, H626* | 10.9 |
| N28W, N196W, N207W, S386D, N603W, H626* | 9.3 |
| R38Y, N196W, H626* | 16.4 |
| N196W, H259Y, H626* | 11.4 |
| N196W, Q412W, H626* | 10 |
| N196W, F212W, H626* | 11.7 |
| N196W, V599W, H626* | 10 |
| N196W, H550Y, P605S, H626* | 13.4 |
| N196W, H550Y, K589F, H626* | 11.7 |
| N196W, H550Y, D608Y, H626* | 11.8 |
| N196W, M574W, L614W, H626* | 14.4 |
| N196W, G533H, M574W, L614W, H626* | 9.5 |
| N196W, V543P, N570H, H626* | 16.8 |
| N196W, G533H, Y575W, L614W, H626* | 11.1 |
| N196W, A545P, T576Y, H626* | 14.4 |
| N196W, A566P, T578Y, H626* | 11.9 |
| N196W, K549Y, G560P, H626* | 15.2 |
| N196W, A566P, L577Y, H626* | 14.4 |
| N196W, I547Y, G560P, H626* | 13.2 |
| N196W, M574MW, H626* | 16.5 |
| N196W, K549*, H550*, D551*, M574MW, P580*, E581*, N582*, F592FK , | 13.1 |
| H626* | |
| N196W, L614W, G619W, H626* | 14.4 |
| N28W, I108P, N196W, N207W, S386D, A466V, Q542K, N603W, H626* | 19.5 |
| N28W, I108P, N196W, N207W, S386D, N603W, H626* | 16.2 |
| N196W, D282P, D283*, H626* | 7.7 |
| W138Y, N196W, H626* | 6.6 |
| N28W, N196W, N207W, S386D, N603W, H626* | 19.5 |
| N196W, S388W, A424P, H626* | 15.9 |
| N196W, S388W, A424P, L489Q, H626* | 16 |
| A117T, N196W, H550Y, D608Y, H626* | 7.8 |
| N196W, Q457R, Y575W, L614W, H626* | 13.4 |
| N196W, S199G | 41.7 |
| N196W, A222V | 40.4 |
| N196W, A222E | 44.4 |
| N196W, A222I | 32.4 |
| S199G, A222V | 55.6 |
| S199G, A222E | 40.7 |
| S199G, A222I | 48.5 |
| N196W, S199G, A222I | 72.5 |
| Q134L, H626* | Below 5 |
| L150W, N156K, N196W, S199G, A222V | 71.7 |
| L150Y, N156K, N196W, S199G, A222I | 63.2 |
| L150W, N196W, S199G, A222I, A428S | 73.1 |
| L150F, N156K, N196W, S199G, A222I | 67.7 |
| L150Y, N156R, N196W, A222V | 70.6 |
| L150M, N156R, N196W, S199G, A222I | 64.9 |
| L150M, N156R, N196W, A222V | 61.2 |
| L150Y, N156R, N196W, S199G, A222V | 69.8 |
| L150M, N156K, N196W, S199G, A222V | 51.1 |
| L150Y, N156R, N196W, A222I | 94.2 |
| L150H, N156R, N196W, S199G, A222I | 63.1 |
| L150H, N156K, N196W, A222V | 75 |
| L150W, N156R, N196W, A222I | 87.6 |
| L150F, N156R, N196W, A222I | Above 95 |
| L150F, N156K, N196W, S199G, A222V | 81.6 |
| L150H, N156K, N196W, S199G, A222V | 60.8 |
| L150F, N156R, S199G, A222I | 65.9 |
| L150M, N156K, N196W, A222V | 57.5 |
| L150W, N156K, N196W, S199G, A222I | 72.2 |
| N156K, N196W, S199G, A222V | 65.3 |
| L150Y, N156R, S199G, A222I | 88.7 |
| L150M, N156R, S199G, A222I | 64.4 |
| L150W, N156K, S199G, A222V | 74.6 |
| L150W, N156R, N196W, S199G, A222V | 58.4 |
| L150Y, N156R, N196W | 52.1 |
| N156K, N196W, A222V | 59 |
| N156K, N196W, S199G | 46.4 |
| N156R, S199G, A222V | 82.2 |
| S113H, N196W, S199G, A222V | 55.5 |
| Q71E, S113H, N196W, S199G, A222V | 52.9 |
| N196W, S199G, A222V, D283A | 78 |
| N196W, S199G, A222V, D283P | 88.5 |
| W142E, D193SQY, N196W, S199G, A222V, R224K | 13.5 |
| E96K, K101R, L150W, N156R, D179S, N196W, S199G, T208N, A222V | 68.3 |
| E96K, K101R, L150Y, N156R, D179S, N196W, S199G, T208N, A222V, | 72.8 |
| E284Q | |
| E96K, K101R, L150M, N156R, D179S, N196W, S199G, T208N, A222V, | 58.2 |
| E284Q | |
| S113Q, Q134E, N196W, S199G, A222V | 50.5 |
| S113D, Q134N, N196W, S199G, A222V | 67 |
| S113F, N196W, S199G, A222V | 70.5 |
| E171Q, N196W, S199G, N204D, A222V | 81.9 |
| N196W, S199G, A222V, H241N, S245N, T278N, E284Q, E285V | 80 |
| N196W, S199G, A222V, S394K, A414K, K417Y | 56.9 |
| N196W, S199G, A222V, E359Y, S394K, K396S, A414K, K417Y | Above 95 |
| R38H, E96K, G99N, K101R, D179S, N196W, S199G, A222V | 83.1 |
| R38H, E96K, G99N, K101R, D179S, N196W, S199G, A222V, E284Q | 80.3 |
| V107T, H110D, N196W, S199G, A222V | 66.9 |
| Q134T, L150Y, N196W, S199G, A222V | 86.2 |
| S113F, L150W, N196W, S199G, A222V | 68.6 |
| S113F, L150Y, N196W, S199G, A222V | Above 95 |
| E96K, K101R, N156R, D179S, N196W, S199G, T208N, A222V | 69.4 |
| E96K, K101R, L150Y, N156R, D179S, N196W, S199G, T208N, A222V | 61.3 |
| E96K, K101R, L150M, N156R, D179S, N196W, S199G, T208N, A222V | 86.7 |
| E96K, K101R, N156R, D179S, N196W, S199G, T208N, A222V, E284Q | 66.9 |
| E96K, K101R, L150W, N156R, D179S, N196W, S199G, T208N, A222V, | 72.6 |
| E284Q | |
| N28R, Q86R, N196W, S199G, A222V | 63.1 |
| N28R, Q86R, K89R, N196W, S199G, A222V | 91.9 |
| G56P, N196W, S199G, S209L, A222V | 88.2 |
| E96K, N196W, S199G, A222V | 76.4 |
| T10I, N196W, S199G, A222V | 80.7 |
| D39R, N196W, S199G, A222V | 75.8 |
| R64S, N196W, S199G, A222V | 90.4 |
| T10I, D39R, R64S, N196W, S199G, A222V | 78.2 |
| T10I, D39R, N196W, S199G, A222V | 56.8 |
| D39R, E96K, N196W, S199G, A222V | 87 |
| R64S, D90E, E96K, N196W, S199G, A222V | 85.5 |
| R38H, D39R, E96K, G99N, K101R, D179S, N196W, S199G, A222V | 82.9 |
| R38H, R64S, E96K, G99N, K101R, D179S, N196W, S199G, A222V | 90.1 |
| T10I, R38H, R64S, E96K, G99N, K101R, D179S, N196W, S199G, A222V | 47.6 |
| T10I, R38H, R64S, D90E, E96K, G99N, K101R, D179S, N196W, S199G, | 51 |
| A222V | |
| T10I, R38H, D39R, E96K, G99N, K101R, D179S, N196W, S199G, A222V | 65.4 |
| R38H, D39R, R64S, E96K, G99N, K101R, D179S, N196W, S199G, A222V | 53.5 |
| E96K, N196W, S199G, A222V, E284Q | 58.4 |
| T10I, N196W, S199G, A222V, E284Q | 58.6 |
| D39R, N196W, S199G, A222V, E284Q | 50.4 |
| R64S, N196W, S199G, A222V, E284Q | 54.4 |
| T10I, D39R, E96K, N196W, S199G, A222V, E284Q | 62.3 |
| D193SQY, N196W, S199G, A222V | 38 |
| Q134T, N196W, S199G, A222V | 47.8 |
| L174I, N196W, S199G, T208N, A222V | 63.6 |
| Y183F, N196W, S199G, T208S, A222V | 64.8 |
| N127D, N156R, N196W, S199G, A222V | 83.1 |
| Q134T, L150W, N196W, S199G, A222V | 70.6 |
| N57P, N196W, S199G, A222V | 73.1 |
| N196W, S199G, Q200W, A222V | 58.7 |
| T10I, D39R, R64S, E96K, N196W, S199G, A222V, E284Q | 67.2 |
| T10I, D39R, R64S, N196W, S199G, A222V, E284Q | 78.1 |
| T10I, R64S, E96K, N196W, S199G, A222V, E284Q | 62.1 |
| D39R, R64S, D90E, E96K, N196W, S199G, A222V, E284Q | 64.8 |
| T10I, R64S, E96K, N196W, S199G, A222V | 54.5 |
| T10I, D39R, N196W, S199G, A222V, E284Q | 70.2 |
| D39R, E96K, N196W, S199G, A222V, E284Q | 52.3 |
| D39R, R64S, N196W, S199G, A222V, E284Q | 60.7 |
| R64S, E96K, N196W, S199G, A222V, E284Q | 67.9 |
| D39R, R64S, N196W, S199G, A222V | 63 |
| S12*, S13*, V14*, K15*, N16*, I103Y, N196W, S199G, A222V, N233S, | 48.8 |
| T308Y | |
| S12*, S13*, V14*, K15*, N16*, A43D, I103Y, N196W, S199G, A222V, | 49.9 |
| N233S, T308M | |
| V9L, S12P, V14I, N16S, A43T, N196W, S199G, A222V | 70.9 |
| S12*, S13*, V14*, K15*, N16*, N196W, S199G, A222V | 54.1 |
| N28W, N196W, S199G, A222V | 66.8 |
| N196W, S199G, A222V, N392W, K417W | 63.1 |
| T10I, D39R, E96K, N196W, S199G, A222V | 70.7 |
| V9D, R38H, N196W, S199G, A222V, T348K | 70.4 |
| S113F, L150Y, N156K, N196W, S199G, A222V, H626* | 59.2 |
| S113Y, L150Y, N156K, N196W, S199G, A222V, H626* | 73.1 |
| S113W, L150Y, N156K, N196W, S199G, A222V, H626* | 68.4 |
| S113F, N156K, N196W, S199G, A222V, H626* | 51.7 |
| S113Y, N156K, N196W, S199G, A222V, H626* | 42.1 |
| S113W, N156K, N196W, S199G, A222V, H626* | 59.2 |
| W138Y, L150V, N196W, S199G, A222V, H626* | 43.9 |
| W138Y, L150V, D179G, N196W, S199G, A222V, H626* | 39.3 |
| W138Y, L150V, N196W, S199G, L218W, A222V, H626* | 29.8 |
| E96K, Q134L, D179S, N196W, S199G, A222V, E284Q, H626* | 78.5 |
| E96K, Q134L, L150Y, N156R, D179S, N196W, S199G, A222V, E284Q, | 51 |
| H626* | |
| E96K, L150Y, N156R, D179S, N196W, S199G, A222V, E284Q, H626* | 65.2 |
| R38H, E96K, G99N, K101R, Q134L, D179S, N196W, S199G, S221N, | 65.1 |
| A222V, H626* | |
| R38H, E96K, G99N, K101R, Q134L, D179S, N196W, S199G, A222V, H626* | 69.9 |
| R38H, E96K, G99N, K101R, Q134L, L150Y, N156R, D179S, N196W, | 62.1 |
| S199G, A222V, H626* | |
| R38H, E96K, G99N, K101R, L150Y, N156R, D179S, N196W, S199G, | 59.4 |
| A222V, H626* | |
| L150F, N196W, S199G, A222I, H626* | 86.7 |
| Q134L, L150F, N156R, N196W, S199G, A222I, H626* | 78.8 |
| Q134L, L150H, N156R, N196W, S199G, A222I, H626* | 83.7 |
| Q134L, L150M, N156K, N196W, S199G, A222I, H626* | 68.1 |
| Q134L, L150Y, N156K, N196W, S199G, A222I, Q457L, H626* | 80.8 |
| Q134M, L150W, N156K, N196W, S199G, A222I, H626* | 50.9 |
| Q134W, L150W, N156K, N196W, S199G, A222I, H626* | 63.2 |
| L150W, L152M, N156K, N196W, S199G, A222I, H626* | 56.9 |
| S113F, L150W, N156K, N196W, S199G, A222I, H626* | 69.2 |
| S113Y, L150W, N156K, N196W, S199G, A222I, H626* | 55.6 |
| L150W, G151W, N156K, N196W, S199G, A222I, H626* | 23.2 |
| L150W, G151S, N156K, N196W, S199G, A222I, H626* | 22.1 |
| S113F, L150W, G151S, N156K, N196W, S199G, A222I, H626* | 38.3 |
| Y67W, W68Y, L150W, N156K, N196W, S199G, A222I, H626* | 60.6 |
| A43V, L150M, G151F, N156R, N196W, S199G, A222I | 48.2 |
| L150M, G151Y, N156R, N196W, S199G, A222I | 20.2 |
| L150M, G151W, N156R, N196W, S199G, A222I | 25.5 |
| L150M, G151S, N156R, N196W, S199G, A222I, Y534H | 52.8 |
| S113F, L150M, G151S, N156R, N196W, S199G, A222I | 17.6 |
| Q134L, L150F, N156K, N196W, S199G, A222I, H626* | 53.5 |
| L150W, G151F, N156K, N196W, S199G, A222I, H626* | 50.4 |
| L150W, G151Y, N156K, N196W, S199G, A222I, H626* | 46.4 |
| Y67W, W68Y, L150W, N156K, N196W, S199G, A222I | 58.1 |
| G56W, N57P, Y67W, W68Y, L150W, N156K, N196W, S199G, A222I, H626* | 60.5 |
| K54I, Y67W, W68S, S113G, N196W, S199G, A222V, A382T, H626* | 44.3 |
| K54I, Y67W, W68S, S113G, D114Q, L150V, N196W, S199G, A222V, H626* | 24.4 |
| K54I, Y67W, W68S, S113G, W138Y, N196W, S199G, A222V, H626* | 39.6 |
| K54I, Y67W, W68S, S113G, D114Q, W138Y, L150V, N196W, S199G, | 18.8 |
| A222V, H626* | |
| Q134L, N196W, S199G, A222V, H626* | 57.7 |
| V107T, I108L, H110D, F169H, N196W, S199G, A222V, H626* | 69.9 |
| N196W, S199G, A222V, N392W, K417W, H626* | 59.8 |
| V9D, R38H, N196W, S199G, A222V, T348K, H626* | 50.4 |
| E96H, L150Y, N156R, N196W, S199G, A222V, E284Q, H626* | 69.8 |
| Q134L, L150Y, N196W, S199G, A222V, H626* | 70.4 |
| L150Y, N196W, S199G, A222V, H626* | Above 95 |
| Q134L, L150Y, N196W, S199G, A222V, H626* | Above 95 |
| L150F, N156R, N196W, S199G, A222V, H626* | 52.6 |
| L150H, N156R, N196W, S199G, A222V, H626* | 70.1 |
| Q134L, L150F, N156R, N196W, S199G, A222V, H626* | Above 95 |
| Q134L, L150H, N156R, N196W, S199G, A222V, H626* | Above 95 |
| Q134L, L150F, N156K, N196W, S199G, A222V, H626* | Above 95 |
| Q134L, L150H, N156K, N196W, S199G, A222V, H626* | Above 95 |
| Q134L, L150Y, N156K, N196W, S199G, A222V, H626* | Above 95 |
| L150Y, N156K, N196W, S199G, A222V, H626* | Above 95 |
| Q134L, L150Y, N156K, N196W, S199G, A222V, H626* | Above 95 |
| Q134W, L150Y, N156K, N196W, S199G, A222V, H626* | Above 95 |
| Q134M, L150Y, N156K, N196W, S199G, A222V, H626* | Above 95 |
| Q134M, L150Y, N156K, N196W, S199G, A222V, A466V, H626* | Above 95 |
| L150Y, L152M, N156K, N196W, S199G, A222V, H626* | Above 95 |
| L150S, N196W, S199G, A222V, H626* | Above 95 |
| N196W, S199G, A222V, N281S, H626* | Above 95 |
| Y67T, N196W, S199G, A222V, H626* | Above 95 |
| Q71N, N196W, S199G, A222V, H626* | Above 95 |
| Q71N, A94D, N196W, S199G, A222V, H626* | Above 95 |
| N196W, S199G, L218F, A222V, H626* | 64.8 |
| N196W, S199G, L218W, A222V, H626* | Above 95 |
| N196W, S199G, A222V, T278W, H626* | 66.8 |
| N196W, S199G, A222V, T278W, T459M, H626* | Above 95 |
| N196W, S199G, A222V, T278Y, H626* | Above 95 |
| N196W, S199G, A222V, S275N, H626* | 59.8 |
| N196W, S199G, A222V, S275L, H626* | Above 95 |
| N196W, S199G, A222V, S335Q, H626* | Above 95 |
| N196W, S199G, A222V, S335K, H626* | 71.3 |
| N196W, S199G, A222V, S335R, H626* | Above 95 |
| N196W, S199G, L218W, A222V, S335K, H626* | Above 95 |
| N196W, S199G, L218W, A222V, S335Q, H626* | 92.1 |
| Y67W, N196W, S199G, A222V, H626* | Above 95 |
| N196W, S199G, A222V, N281Q, H626* | Above 95 |
| L150M, N156R, N196W, S199G, A222V, H626* | Above 95 |
| Q134L, L150M, N156R, N196W, S199G, A222V, H626* | Above 95 |
| Q134L, L150M, N156K, N196W, S199G, A222V, H626* | Above 95 |
| D39R, N196W, S199G, A222V, N281Q, E284Q | Above 95 |
| D39R, N196W, S199G, A222V, E284Q, Q479QP | Above 95 |
| D39R, Y70F, N196W, S199G, A222V, E284Q | Above 95 |
| N28W, D39R, N196W, S199G, A222V, E284Q | Above 95 |
| D39R, N196W, S199G, N207W, A222V, E284Q | Above 95 |
| E1*, T2*, A3*, N4*, K5*, S6*, N7*, K8*, D39R, N196W, S199G, N207W, | Above 95 |
| A222V, E284Q, H626* | |
| N196W, N207W | 19.2 |
| N196W, N207W, E284Q | 19 |
| E1*, T2*, A3*, N4*, K5*, S6*, N7*, K8*, N196W, N207W, E284Q | 16.8 |
| L150Y, N196W, S199G, A222V, H626* | Above 95 |
| E1*, T2*, A3*, N4*, K5*, S6*, N7*, K8*, L150Y, N196W, S199G, A222V | Above 95 |
| E96K, K101R, L150M, N156R, D179S, N196W, S199G, N207W, T208N, | Above 95 |
| A222V | |
| E1*, T2*, A3*, N4*, K5*, S6*, N7*, K8*, E96K, K101R, L150M, N156R, | Above 95 |
| D179S, N196W, S199G, T208N, A222V | |
| Q134L, L150H, N156R, N196W, S199G, A222I, E284Q, H626* | Above 95 |
| E1*, T2*, A3*, N4*, K5*, S6*, N7*, K8*, Q134L, L150H, N156R, N196W, | Above 95 |
| S199G, A222I, H626* | |
| E1*, T2*, A3*, N4*, K5*, S6*, N7*, K8*, Q134L, L150H, N156R, N196W, | Above 95 |
| S199G, N207W, A222I, E284Q | |
| E96K, D179S, N196W, S199G, A222V, E284Q, H626* | Above 95 |
| E1*, T2*, A3*, N4*, K5*, S6*, N7*, K8*, E96K, D179S, N196W, S199G, | 88.4 |
| A222V, E284Q | |
| R64S, E96K, N196W, S199G, N207W, A222V | Above 95 |
| N28W, D39R, N196W, S199G, N207W, A222V, E284Q | Above 95 |
| D39R, N196W, S199G, A222V, E284Q, H626* | Above 95 |
| D39R, N196W, S199G, N207W, A222V, E284Q | Above 95 |
| E1*, T2*, A3*, N4*, K5*, S6*, N7*, K8*, D39R, N196W, S199G, A222V, | Above 95 |
| E284Q | |
| E1*, T2*, A3*, N4*, K5*, S6*, N7*, K8*, N196W, N207W, E511D | 20.9 |
| L150Y, N196W, S199G, N207W, A222V | Above 95 |
| E96K, K101R, L150M, N156R, D179S, N196W, S199G, T208N, A222V, | Above 95 |
| H626* | |
| E96K, K101R, L150M, N156R, D179S, N196W, S199G, T208N, A222V, | Above 95 |
| E284Q | |
| E1*, T2*, A3*, N4*, K5*, S6*, N7*, K8*, E96K, K101R, L150M, N156R, | Above 95 |
| D179S, N196W, S199G, N207W, T208N, A222V, E284Q, H626* | |
| Q134L, L150H, N156R, N196W, S199G, A222I | Above 95 |
| N28W, E96K, D179S, N196W, S199G, A222V, E284Q | Above 95 |
| R64S, E96K, N196W, S199G, A222V, H626* | Above 95 |
| R64S, E96K, N196W, S199G, A222V, E284Q | Above 95 |
| E1*, T2*, A3*, N4*, K5*, S6*, N7*, K8*, R64S, E96K, N196W, S199G, A222V | 93.7 |

### Example 9—Evaluation of Selected Variants of the Invention in Raw Starch to Ethanol Process

### Materials & Methods

Saccharomyces cerevisiae strain MBG5012: Saccharomyces cerevisiae strain MBG5012 (deposited under Accession No. NRRL Y67549 at the Agricultural Research Service Patent Culture Collection (NRRL), Northern Regional Research Center, 1815 University Street, Peoria, Ill., USA).

Variant A: SEQ ID NO 1 containing mutations N196W and H626*

Variant B: SEQ ID NO 1 containing mutations E96K, D179S, N196W, S199G, A222V and E284Q

Variant C: SEQ ID NO 1 containing mutations R64S, E96K, N196W, S199G, A222V

Variant D: SEQ ID NO 1 containing the mutations N196W, N207W and H626*

Variant E: SEQ ID NO 1 containing mutations S199G and H626*

Variant F: SEQ ID NO 1 containing mutations L150Y, N196W, S199G, A222V

Glucoamylase A: A glucoamylase from Trametes cingulata disclosed as SEQ ID NO: 4.

Saccharification enzyme blend: An enzyme blend comprising a glucoamylase SEQ ID NO: 4 and a fungal acid alpha-amylase disclosed as SEQ ID NO: 5.

### Example 9a. Evaluation of Effect of Alpha-Amylase Variant a on Ethanol and Residual Starch Following Simultaneous Saccharification and Fermentation (SSF) of Raw Starch Corn Mash

This example describes the evaluation of the alpha-amylase Variant A compared to SEQ ID NO for improved residual starch and ethanol following an 88-hour raw starch corn mash SSF.

### Seed Culture

Cryo-preserved culture of fermenting organism Saccharomyces cerevisiae strain MBG5012 was grown in liquid YPD media (Yeast extract, 10 g. Peptone, 20 g. Dextrose, 60 g. dissolve in 1 L of distilled water). Cultivation was done aseptically in a sterile 125-m1 Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed culture (40 ml) was centrifuged at 3,500 rpm for 10 min at 22° C., and the resulting cell pellet was washed and resuspended in tap water. The resuspended cells were used to inoculate the corn mash at the beginning of SSF.

### Corn Mash

Corn flour and backset from a raw starch ethanol plant was mixed. The corn slurry was supplemented with 200 ppm urea and 3 ppm of antibiotic LACTROL™ and its pH was adjusted to 4.5 prior to use in SSF. Final dried solids level was determined to be 37.2% DS.

### Simultaneous Saccharification and Fermentation (SSF)

All fermentations were carried out in 125 mL jars with caps having a drilled hole. Jars were filled with 70-85 g of corn slurry and inoculated with seed culture at 10 million cells per gram mash. Glucoamylase A (SEQ ID NO: 4) was added to the jars at 88 μg enzyme protein per g of dry corn solids. Alpha-amylases were added to the jars at 32 μg enzyme protein per g of dry corn solids. All jars contained the same glucoamylase and either alpha-amylase with SEQ ID NO 1 or Variant A. Jars were incubated in a water bath at 32° C. Jars were swirled two times per day. Fermentation was run for 88 hours.

### Ethanol, Sugars and Organic Acid Analyses

After 88 h of fermentation, 4 mL samples were taken from each jar, and 100 uL of 40% (v/v) H₂SO₄ was added to stop the reaction. These mixtures were vortexed, and centrifuged at 3,000 rpm for 10 min at 22° C. The resulting supernatant was filtered through a 0.2 μm syringe filter. Filtered samples were stored at 4° C. prior to and during HPLC analysis. Analysis of ethanol, sugars and organic acids was conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and analytes were detected using a Refractive Index detector at 55° C.

For the residual starch assay, starch is considered insoluble in aqueous solution, so any soluble glucose/maltodextrin is removed via two additional washes using deionized water. Washed insoluble solid was freeze dried and 0.1 g of this dried material was then subjected to the assay (described below) to obtain the starch of the insoluble solid. The total dry solid was determined in order to convert the starch of insoluble solid to the starch of dry solid. Residual starch level, when treated with various alpha-amylases, can be compared to the ethanol titer to determine the overall effectiveness of alpha-amylases in accessing starch and converting that to ethanol in RSH SSF.

### Total Dry Solid Determination

Total dry solid was determined on the 88 h samples by placing ˜1.5 g of fermentation sample (wet weight) onto a pre-weighed aluminum weigh boat and incubated in a forced air oven maintaining at 105° C. for 16-20 hours. The percent of total dry solid was calculated by dividing the dried weight to the wet weight of the sample.

### The Insoluble Solid Determination

The insoluble solid was determined on the 88 h samples by placing between 7-10 grams of fermentation sample into a pre-weighed 15 mL Genogrind vials. Deionized water was added to each vial to the 15 mL total volume and then centrifuged at 3500 rpm for 10 minutes. The supernatant was decanted and the insoluble solid washed two more times by resuspending in deionized (to 15 mL), followed by centrifugation and decanting. The insoluble pellet was placed in −20° C. freezer for >2 hours and then lyophilized for 24 hours using Labconco FreeZone freeze dryer. The weight of the freeze dried sample was measured and the percent insoluble solid was calculated by dividing the freeze dried weight by the wet mash weight.

### Residual Starch Assay

Residual starch is determined following the Megazyme Total Starch (AA/AMG) Kit (Catalog #K-TSTA) with the following modification. The freeze-dried sample (from the insoluble solid determination step) was ground to fine powder using the Geno/Grinder Spex Sample Prep at 1600 rpm for 4 minutes. The starch level in this ground sample (insoluble solid) was determined according to the method described in the “Example A” of the kit. After all enzyme treatments, the glucose level was quantified following the same HPLC method described in “Ethanol, sugars and organic acid analyses” section. Using the percent of insoluble solids and total dry solid, the starch of insoluble solid was converted to the percent starch of dry solid following the calculation described in the Megazyme kit. The value resulting from the calculation is described as “% residual starch” in result section below.

### Results

FIG. 1 shows the ethanol following 88 hours of fermentation by alpha-amylase treatment for Variant A and SEQ ID NO 1. FIG. 2 shows the percent residual starch after 88 hours of fermentation by alpha-amylase treatment for Variant A and SEQ ID NO 1. Results indicate improved ethanol and lowered residual starch for fermentations with alpha-amylase Variant A compared to fermentations with SEQ ID NO 1.

### Example 9b. Evaluation of Effect of Alpha-Amylases Variant B and Variant C on Ethanol and Residual Starch Following SSF of Raw Starch Corn Mash

This example describes the evaluation of the alpha-amylases Variant B and Variant C compared to SEQ ID NO 1 for improved residual starch and ethanol following an 88-hour raw starch corn mash SSF.

### Seed Culture

Same as described in Example 1.

### Corn Mash

Corn flour and backset from a raw starch ethanol plant was mixed. The corn slurry was supplemented with 200 ppm urea and 3 ppm of antibiotic LACTROL™ and its pH was adjusted to 4.5 prior to use in SSF. Final dried solids level was determined to be 37.9% DS.

### Simultaneous Saccharification and Fermentation (SSF)

Same as described in Example 1.

### Ethanol, Sugars and Organic Acid Analyses

Same as described in Example 1.

### Residual Starch Analysis

For this assay, starch is considered insoluble in aqueous solution, so any soluble glucose/maltodextrin was removed via two washes using deionized water. The insoluble solid that was sampled from the fermentation was pelleted by centrifugation at 3500 rpm for 10 minutes at 22° C. After removing supernatant, the pellet was washed with deionized water two times, centrifuged and supernatant was removed. The insoluble solid was resuspended in 4 mL deionized water and 0.05 mL of Saccharification enzyme blend (SEQ ID NO: 4 and SEQ ID NO: 5). The mixture was vortexed and incubated for 26 hours at 50° C. Samples were centrifuged at 3,500 rpm for 10 min at 22° C. The resulting supernatant was filtered through a 0.2 μm syringe filter. Filtered samples were stored at 4° C. prior to and during HPLC analysis. Analysis of glucose was conducted using the same HPLC analysis as described in Example 1 above (under the Ethanol, sugars and organic acid analysis section). The starch was calculated based on resulting HPLC glucose value and using the 0.9 gram of starch/g glucose conversion factor. The starch value was then divided by the grams of corn mash amount that was sampled into tube at the end of fermentation, resulting in a residual g starch/g mash value. The residual starch level, when treated with various alpha-amylases, can be compared amongst enzyme treatments to determine the overall effectiveness of alpha-amylases in accessing starch.

### Results

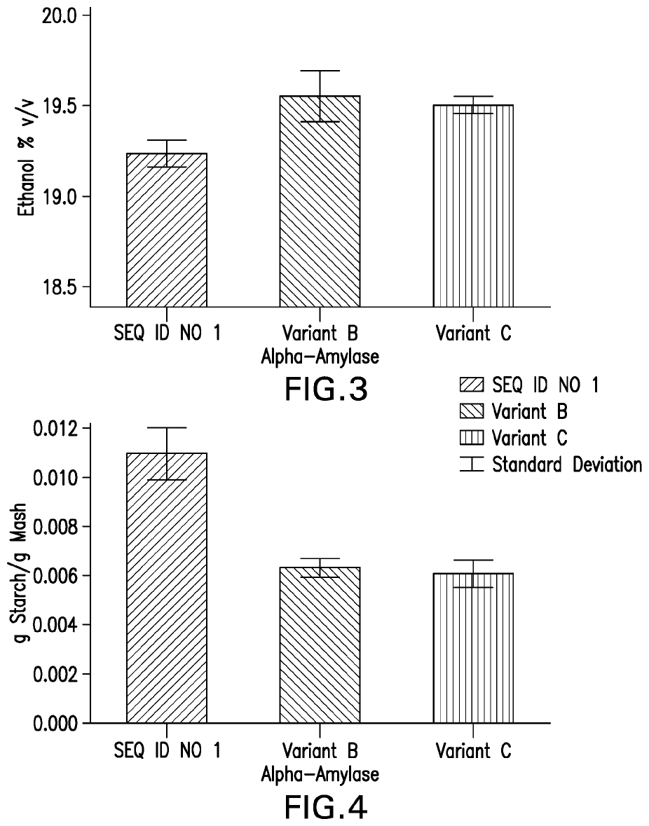

FIG. 3 shows the ethanol following 88 hours of fermentation by alpha-amylase treatment for Variant B, Variant C and SEQ ID NO 1. FIG. 4 shows the g starch/g mash after 88 hours of fermentation by alpha-amylase treatment for Variant B, Variant C and SEQ ID NO 1. Results indicate improved ethanol and lowered residual starch for fermentations with alpha-amylases Variant B or Variant C compared to fermentations with SEQ ID NO 1.

### Example 9c. Evaluation of Effect of Alpha-Amylases Variant D, Variant E and Variant F on Ethanol Following SSF of Raw Starch Corn Mash

This example describes the evaluation of the alpha-amylases Variant D, Variant E and Variant F compared to SEQ ID NO 1 for improved ethanol following a 72-hour raw starch corn mash SSF.

### Seed Culture

Same as described in Example 1.

### Corn Mash

Corn kernels were ground using the Turkish grind setting on a Bunn Coffee Grinder. The % dried solids (DS) of the corn flour was 84.50%. Using the Turkish ground corn and tap water, two corn slurries were prepared. Each corn slurry was supplemented with 1000 ppm urea and 3 ppm of antibiotic LACTROL™ and the pH was adjusted to 4.5 prior to use in SSF. Final dried solids level was determined to be 34.2% DS and 35% DS, respectively.

### Simultaneous Saccharification and Fermentation (SSF)

All fermentations were carried out in 12 mL round-bottom tubes with caps having a drilled hole. Tubes were filled with 3.8-4.5 g of corn slurry and inoculated with seed culture at 10 million cells per gram mash. Glucoamylase A (SEQ ID NO: 4) was added to the tubes at 88 μg enzyme protein per g of dry corn solids. The alpha-amylases were added to the tubes at 32 μg enzyme protein per g of dry corn solids. Variant D, Variant E and SEQ ID NO 1 alpha-amylases were dosed into slurry with starting % DS of 34.2%. Variant F and SEQ ID NO 1 alpha-amylases were dosed into slurry with starting % DS of 35%. All tubes contained the same glucoamylase and one of the alpha-amylases was added per tube. Tubes were incubated in an incubator at 32° C. Tubes were vortexed two times per day. Fermentations were run for 72 hours.

### Ethanol Analysis

After 72 hours, 100 μL of 40% v/v H₂SO₄ was added to each sample tube, samples were vortexed, and centrifuged at 3,000 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

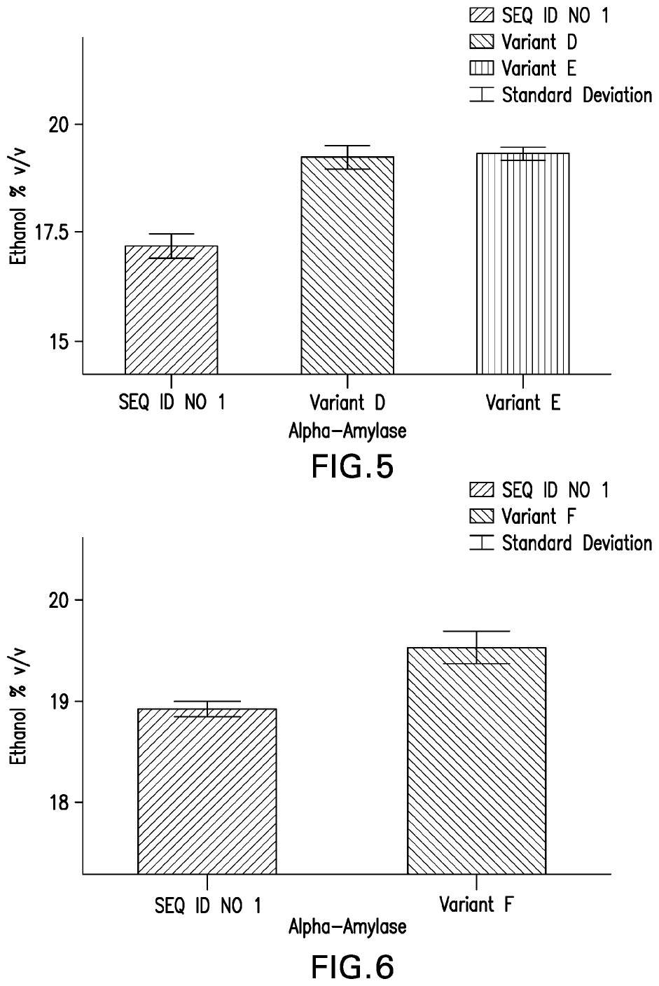

FIG. 5 shows ethanol after 72 hours of fermentation for treatments with SEQ ID NO 1, Variant D and Variant E. FIG. 6 shows ethanol after 72 h for fermentation for treatments with SEQ ID NO 1 and Variant F. Results indicate improved ethanol for fermentations with alpha-amylases Variant D, Variant E or Variant F compared to fermentations with SEQ ID NO 1.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A process of producing a fermentation product from raw starch material, comprising the steps of: (a) saccharifying starch-containing material at a temperature below the initial gelatinization temperature of said starch-containing material; and (b) fermenting with a fermenting organism, wherein step (a) is carried out in the presence of at least a variant alpha-amylase comprising an alteration at one or more positions corresponding to positions 196, 1, 2, 3, 4, 5, 6, 7, 8, 9, 10, 11, 12, 13, 14, 15, 16, 28, 38, 39, 43, 54, 56, 57, 64, 67, 68, 70, 71, 86, 89, 90, 94, 96, 99, 101, 103, 107, 108, 110, 113, 114, 117, 127, 134, 138, 142, 150, 151, 152, 156, 169, 171, 174, 179, 183, 193, 199, 200, 204, 205, 207, 208, 209, 212, 218, 221, 222, 224, 233, 241, 245, 259, 275, 278, 281, 282, 283, 284, 285, 308, 323, 335, 348, 359, 382, 386, 388, 392, 394, 396, 412, 414, 417, 424, 428, 457, 459, 466, 479, 489, 511, 533, 534, 542, 543, 545, 547, 549, 550, 551, 560, 566, 570, 574, 575, 576, 577, 578, 580, 581, 582, 589, 592, 599, 603, 605, 608, 614, 619, or 626 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, and optionally a glucoamylase.

2. The process according to claim 1, wherein the variant alpha-amylase comprises a substitution at one or more positions corresponding to positions 64, 96,150, 179, 196, 199, 207, 222, 284 and 603 of the polypeptide of SEQ ID NO: 1, wherein the variant has alpha-amylase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

3. The process of claim 1, wherein the variant alpha-amylase comprises an alteration selected from the group consisting of: E1*, T2*, A3*, N4*, K5*, S6*, N7*, K8*, V9*, V9D, V9L, T10*, T10I, A11*, S12*, 512P, S13*, V14*, V14I, K15*, N16*, N165, N28R, N28W, R38H, R38Y, D39R, A43D, A43T, A43V, K54I, G56P, G56W, N57P, R64S, Y67T, Y67W, W68S, W68Y, Y70F, Q71E, Q71N, Q86R, K89R, D90E, A94D, E96H, E96K, G99N, K101R, 1103Y, V107T, 1108L, 1108P, H110D, S113D, S113F, S113G, S113H, S113Q, S113W, S113Y, D114Q, A117T, N127D, Q134E, Q134L, Q134M, Q134N, Q134T, Q134W, W138Y, W142E, L150F, L150H, L150M, L150S, L150V, L150W, L150Y, G151F, G151S, G151W, G151Y, L152M, N156K, N156R, F169H, E171Q, L174I, D179G, D179S, Y183F, Y183I, D193SQY, N196W, S199G, Q200W, N204D, 1205Y, N207W, T208N, T208S, S209L, F212W, L218F, L218W, S221N, A222E, A222I, A222V, R224K, N233S, H241N, S245N, H259Y, S275L, S275N, T278N, T278W, T278Y, N281Q, N281S, D282P, D283*, D283A, D283P, E284Q, E285V, T308M, T308Y, R323K, S335K, S335Q, S335R, T348K, E359Y, A382T, S386D, S388W, N392R, N392W, S394K, K396S, Q412W, A414K, K417W, K417Y, A424P, A428S, Q457L, Q457R, T459M, A466V, Q479QP, L489Q, E511D, G533H, Y534H, Q542K, V543P, A545P, 1547Y, K549*, K549Y, H550*, H550Y, D551*, G560P, A566P, N570H, M574MW, M574W, Y575W, T576Y, L577Y, T578Y, P580*, E581*, N582*, K589F, F592FK, V599W, N603W, P605S, D608Y, L614W, G619W, and H626*.

4. The process of claim 2, wherein the variant alpha-amylase comprises a substitution selected from the group consisting of: R64S, E96K, L150Y, L150W, L150H, L150M, L150F, D179S, N196W, S199G, N207W, A222E, A222I, A222V, E284Q and N603W.

5. The process of claim 1, wherein the variant alpha-amylase has an improved property relative to a parent alpha amylase, wherein the improved property is increased pH stability at pH 4.0, 32° C. to 37° C., compared to the alpha-amylase disclosed as SEQ ID NO: 1.

6. The process of claim 1, wherein the variant alpha-amylase comprises a substitution or a combination of substitutions selected from:
A222I;
A222V;
A222E;
S199G;
N196W;
N207W;
N603W:
L150Y;
L150W;
L150H;
L150M;
L150F;
R64S:
E96K;
D179S;
E284Q;
N207W+N603W;
N196W+N207W;
N196W+N603W;
N196W+N207W+N603W;
A222I+S199G+N196W;
A222V+S199G+N196W;
A222E+S199G+N196W;
A222V+S199G+N196W+L150Y;
L150H+S199G+A222I;
L150M+S199G+A222V;
N196W+S199G+A222V+N603W;
L150F+N196W+S199G+A222I;
L150M+N196W+S199G+A222V;
L150W+S199G+A222V;
L150H+N196W+S199G+A222V;
L150W+N196W+S199G+A222I;
L150Y+S199G+A222V;
E96K+D179S+N196W+S199G+A222V+E284Q;
R64S+E96K+N196W+S199G+A222V;
wherein the variant has alpha-amylase activity and wherein the variant has at least 60%, at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

7. The process according to claim 1, wherein the variant alpha-amylase comprises an N-terminal deletion comprising at least amino acids 11 to 626 of SEQ ID NO: 1, at least amino acids 12 to 626 of SEQ ID NO: 1, or at least amino acids 13 to 626 of SEQ ID NO: 1.

8. The process according to claim 1, wherein the variant alpha-amylase further comprises a C-terminal deletion, H626*.

9. The process of claim 1, wherein the variant alpha-amylase comprises an alteration or a combination of alterations selected from:
N28W;
N196W;
S199G;
N196W+V599W;
N196W+H550Y+P605S;
N196W+A545P+T576Y;
N196W+K549Y+G560P;
I108P+Y183I+N196W+1205Y;
N196W+R323K;
N196W+D283P;
W138Y+N196W;
L150W;
N196W+N392W+K417W;
N196W+N392R+K417W;
N196W+K549*+H550*+D551*;
N196W+P580*+E581*+N582*;
N196W+F592FK;
N28W+N196W+N207W+S386D+N603W;
R38Y+N196W;
N196W+H259Y;
N196W+Q412W;
N196W+F212W;
N196W+V599W;
N196W+H550Y+P605S;
N196W+H550Y+K589F;
N196W+H550Y+D608Y;
N196W+M574W+L614W;
N196W+G533H+M574W+L614W;
N196W+V543P+N570H;
N196W+G533H+Y575W+L614W;
N196W+A545P+T576Y;
N196W+A566P+T578Y;
N196W+K549Y+G560P;
N196W+A566P+L577Y;
N196W+I547Y+G560P;
N196W+M574MW;
N196W+K549*+H550*+D551*+M574MW+P580*+E581*+N582*+F592FK;
N196W+L614W+G619W;
N28W+I108P+N196W+N207W+S386D+A466V+Q542K+N603W;
N28W+I108P+N196W+N207W+S386D+N603W;
N196W+D282P+D283*;
W138Y+N196W;
N28W+N196W+N207W+S386D+N603W;
N196W+S388W+A424P;
N196W+S388W+A424P+L489Q;
A117T+N196W+H550Y+D608Y;
N196W+Q457R+Y575W+L614W;
N196W+S199G;
N196W+A222V;
N196W+A222E;
N196W+A222I;
S199G+A222V;
S199G+A222E;
S199G+A222I;
N196W+S199G+A222I;
Q134L;
L150W+N156K+N196W+S199G+A222V;
L150Y+N156K+N196W+S199G+A222I;
L150W+N196W+S199G+A222I+A428S;
L150F+N156K+N196W+S199G+A222I;
L150M+N156R+N196W+S199G+A222I;
L150M+N156R+N196W+A222V;
L150Y+N156R+N196W+S199G+A222V;
L150M+N156K+N196W+S199G+A222V;
L150Y+N156R+N196W+A222I;
L150H+N156R+N196W+S199G+A222I;
L150H+N156K+N196W+A222V;
L150W+N156R+N196W+A222I;
L150F+N156R+N196W+A222I;
L150F+N156K+N196W+S199G+A222V;
L150H+N156K+N196W+S199G+A222V;
L150F+N156R+S199G+A222I;
L150M+N156K+N196W+A222V;
L150W+N156K+N196W+S199G+A222I;
N156K+N196W+S199G+A222V;
L150Y+N156R+S199G+A222I;
L150M+N156R+S199G+A222I;
L150W+N156K+S199G+A222V;
L150W+N156R+N196W+S199G+A222V;
L150Y+N156R+N196W;
N156K+N196W+A222V;
N156K+N196W+S199G;
N156R+S199G+A222V;
S113H+N196W+S199G+A222V;
Q71E+S113H+N196W+S199G+A222V;
N196W+S199G+A222V+D283A;
N196W+S199G+A222V+D283P;
W142E+D193SQY+N196W+S199G+A222V+R224K;
E96K+K101R+L150W+N156R+D179S+N196W+S199G+T208N+A222V;
E96K+K101R+L150Y+N156R+D179S+N196W+S199G+T208N+A222V+E284Q;
E96K+K101R+L150M+N156R+D179S+N196W+S199G+T208N+A222V+E284Q;
S113Q+Q134E+N196W+S199G+A222V;
S113D+Q134N+N196W+S199G+A222V;
S113F+N196W+S199G+A222V;
E171Q+N196W+S199G+N204D+A222V;
N196W+S199G+A222V+H241N+S245N+T278N+E284Q+E285V;
N196W+S199G+A222V+5394K+A414K+K417Y;
N196W+S199G+A222V+E359Y+5394K+K396S+A414K+K417Y;
R38H+E96K+G99N+K101R+D179S+N196W+S199G+A222V;
R38H+E96K+G99N+K101R+D179S+N196W+S199G+A222V+E284Q;
V107T+H110D+N196W+S199G+A222V;
Q134T+L150Y+N196W+S199G+A222V;
S113F+L150W+N196W+S199G+A222V;
S113F+L150Y+N196W+S199G+A222V;
E96K+K101R+N156R+D179S+N196W+S199G+T208N+A222V;
E96K+K101R+L150Y+N156R+D179S+N196W+S199G+T208N+A222V;
E96K+K101R+L150M+N156R+D179S+N196W+S199G+T208N+A222V;
E96K+K101R+N156R+D179S+N196W+S199G+T208N+A222V+E284Q;
E96K+K101R+L150W+N156R+D179S+N196W+S199G+T208N+A222V+E284Q;
N28R+Q86R+N196W+S199G+A222V;
N28R+Q86R+K89R+N196W+S199G+A222V;
G56P+N196W+S199G+5209L+A222V;
E96K+N196W+S199G+A222V;
T10I+N196W+S199G+A222V;
D39R+N196W+S199G+A222V;
R64S+N196W+S199G+A222V;
T10I+D39R+R64S+N196W+S199G+A222V;
T10I+D39R+N196W+S199G+A222V;
D39R+E96K+N196W+S199G+A222V;
R64S+D90E+E96K+N196W+S199G+A222V;
R38H+D39R+E96K+G99N+K101R+D179S+N196W+S199G+A222V;
R38H+R64S+E96K+G99N+K101R+D179S+N196W+S199G+A222V;
T10I+R38H+R64S+E96K+G99N+K101R+D179S+N196W+S199G+A222V;
T10I+R38H+R64S+D90E+E96K+G99N+K101R+D179S+N196W+S199G+A222V;
T10I+R38H+D39R+E96K+G99N+K101R+D179S+N196W+S199G+A222V;
R38H+D39R+R64S+E96K+G99N+K101R+D179S+N196W+S199G+A222V;
E96K+N196W+S199G+A222V+E284Q;
T10I+N196W+S199G+A222V+E284Q;
D39R+N196W+S199G+A222V+E284Q;
R64S+N196W+S199G+A222V+E284Q;
T10I+D39R+E96K+N196W+S199G+A222V+E284Q;
D193SQY+N196W+S199G+A222V;
Q134T+N196W+S199G+A222V;
L174I+N196W+S199G+T208N+A222V;
Y183F+N196W+S199G+T208S+A222V;
N127D+N156R+N196W+S199G+A222V;
Q134T+L150W+N196W+S199G+A222V;
N57P+N196W+S199G+A222V;
N196W+S199G+Q200W+A222V;
T10I+D39R+R64S+E96K+N196W+S199G+A222V+E284Q;
T10I+D39R+R64S+N196W+S199G+A222V+E284Q;
T10I+R64S+E96K+N196W+S199G+A222V+E284Q;
D39R+R64S+D90E+E96K+N196W+S199G+A222V+E284Q;
T10I+R64S+E96K+N196W+S199G+A222V;
T10I+D39R+N196W+S199G+A222V+E284Q;
D39R+E96K+N196W+S199G+A222V+E284Q;
D39R+R64S+N196W+S199G+A222V+E284Q;
R64S+E96K+N196W+S199G+A222V+E284Q;
D39R+R64S+N196W+S199G+A222V;
S12*+S13*+V14*+K15*+N16*+1103Y+N196W+S199G+A222V+N233S+T308Y;
S12*+S13*+V14*+K15*+N16*+A43D+1103Y+N196W+S199G+A222V+N233S+T308M;
V9L+S12P+V14I+N16S+A43T+N196W+S199G+A222V;
S12*+S13*+V14*+K15*+N16*+N196W+S199G+A222V;
N28W+N196W+S199G+A222V;
N196W+S199G+A222V+N392W+K417W;
T10I+D39R+E96K+N196W+S199G+A222V;
V9D+R38H+N196W+S199G+A222V+T348K;
S113F+L150Y+N156K+N196W+S199G+A222V;
S113Y+L150Y+N156K+N196W+S199G+A222V;
S113W+L150Y+N156K+N196W+S199G+A222V;
S113F+N156K+N196W+S199G+A222V;
S113Y+N156K+N196W+S199G+A222V;
S113W+N156K+N196W+S199G+A222V;
W138Y+L150V+N196W+S199G+A222V;
W138Y+L150V+D179G+N196W+S199G+A222V;
W138Y+L150V+N196W+S199G+L218W+A222V;
E96K+Q134L+D179S+N196W+S199G+A222V+E284Q;
E96K+Q134L+L150Y+N156R+D179S+N196W+S199G+A222V+E284Q;
E96K+L150Y+N156R+D179S+N196W+S199G+A222V+E284Q;
R38H+E96K+G99N+K101R+Q134L+D179S+N196W+S199G+S221N+A222V;
R38H+E96K+G99N+K101R+Q134L+D179S+N196W+S199G+A222V;
R38H+E96K+G99N+K101R+Q134L+L150Y+N156R+D179S+N196W+S199G+A222V;
R38H+E96K+G99N+K101R+L150Y+N156R+D179S+N196W+S199G+A222V;
L150F+N196W+S199G+A222I;
Q134L+L150F+N156R+N196W+S199G+A222I;
Q134L+L150H+N156R+N196W+S199G+A222I;
Q134L+L150M+N156K+N196W+S199G+A222I;
Q134L+L150Y+N156K+N196W+S199G+A222I+Q457L;
Q134M+L150W+N156K+N196W+S199G+A222I;
Q134W+L150W+N156K+N196W+S199G+A222I;
L150W+L152M+N156K+N196W+S199G+A222I;
S113F+L150W+N156K+N196W+S199G+A222I;
S113Y+L150W+N156K+N196W+S199G+A222I;
L150W+G151W+N156K+N196W+S199G+A222I;
L150W+G151S+N156K+N196W+S199G+A222I;
S113F+L150W+G151S+N156K+N196W+S199G+A222I;
Y67W+W68Y+L150W+N156K+N196W+S199G+A222I;
A43V+L150M+G151F+N156R+N196W+S199G+A222I;
L150M+G151Y+N156R+N196W+S199G+A222I;
L150M+G151W+N156R+N196W+S199G+A222I;
L150M+G151S+N156R+N196W+S199G+A222I+Y534H;
S113F+L150M+G151S+N156R+N196W+S199G+A222I;
Q134L+L150F+N156K+N196W+S199G+A222I;
L150W+G151F+N156K+N196W+S199G+A222I;
L150W+G151Y+N156K+N196W+S199G+A222I;
Y67W+W68Y+L150W+N156K+N196W+S199G+A222I;
G56W+N57P+Y67W+W68Y+L150W+N156K+N196W+S199G+A222I;
K54I+Y67W+W68S+S113G+N196W+S199G+A222V+A382T;
K54I+Y67W+W68S+S113G+D114Q+L150V+N196W+S199G+A222V;
K54I+Y67W+W68S+S113G+W138Y+N196W+S199G+A222V;
K54I+Y67W+W68S+S113G+D114Q+W138Y+L150V+N196W+S199G+A222V;
Q134L+N196W+S199G+A222V;
V107T+I108L+H110D+F169H+N196W+S199G+A222V;
N196W+S199G+A222V+N392W+K417W;
V9D+R38H+N196W+S199G+A222V+T348K;
E96H+L150Y+N156R+N196W+S199G+A222V+E284Q;
Q134L+L150Y+N196W+S199G+A222V;
L150Y+N196W+S199G+A222V;
Q134L+L150Y+N196W+S199G+A222V;
L150F+N156R+N196W+S199G+A222V;
L150H+N156R+N196W+S199G+A222V;
Q134L+L150F+N156R+N196W+S199G+A222V;
Q134L+L150H+N156R+N196W+S199G+A222V;
Q134L+L150F+N156K+N196W+S199G+A222V;
Q134L+L150H+N156K+N196W+S199G+A222V;
Q134L+L150Y+N156K+N196W+S199G+A222V;
L150Y+N156K+N196W+S199G+A222V;
Q134L+L150Y+N156K+N196W+S199G+A222V;
Q134W+L150Y+N156K+N196W+S199G+A222V;
Q134M+L150Y+N156K+N196W+S199G+A222V;
Q134M+L150Y+N156K+N196W+S199G+A222V+A466V;
L150Y+L152M+N156K+N196W+S199G+A222V;
L150S+N196W+S199G+A222V;
N196W+S199G+A222V+N281S;
Y67T+N196W+S199G+A222V;
Q71N+N196W+S199G+A222V;
Q71N+A94D+N196W+S199G+A222V;
N196W+S199G+L218F+A222V;
N196W+S199G+L218W+A222V;
N196W+S199G+A222V+T278W;
N196W+S199G+A222V+T278W+T459M;
N196W+S199G+A222V+T278Y;
N196W+S199G+A222V+S275N;
N196W+S199G+A222V+S275L;
N196W+S199G+A222V+S335Q;
N196W+S199G+A222V+S335K;
N196W+S199G+A222V+S335R;
N196W+S199G+L218W+A222V+S335K;
N196W+S199G+L218W+A222V+S335Q;
Y67W+N196W+S199G+A222V;
N196W+S199G+A222V+N281Q;
L150M+N156R+N196W+S199G+A222V;
Q134L+L150M+N156R+N196W+S199G+A222V;
Q134L+L150M+N156K+N196W+S199G+A222V;
D39R+N196W+S199G+A222V+N281Q+E284Q;
D39R+N196W+S199G+A222V+E284Q+Q479QP;
D39R+Y70F+N196W+S199G+A222V+E284Q;
N28W+D39R+N196W+S199G+A222V+E284Q;
D39R+N196W+S199G+N207W+A222V+E284Q;
E1*+T2*+A3*+N4*+K5*+S6*+N7*+K8*+D39R+N196W+S199G+N207W+A222V+E284Q;
N196W+N207W;
N196W+N207W+E284Q;
E1*+T2*+A3*+N4*+K5*+S6*+N7*+K8*+N196W+N207W+E284Q;
L150Y+N196W+S199G+A222V;
E1*+T2*+A3*+N4*+K5*+S6*+N7*+K8*+L150Y+N196W+S199G+A222V;
E96K+K101R+L150M+N156R+D179S+N196W+S199G+N207W+T208N+A222V;
E1*+T2*+A3*+N4*+K5*+S6*+N7*+K8*+E96K+K101R+L150M+N156R+D179S+N196W+S199G+T208N+A222V;
Q134L+L150H+N156R+N196W+S199G+A222I+E284Q;
E1*+T2*+A3*+N4*+K5*+S6*+N7*+K8*+Q134L+L150H+N156R+N196W+S199G+A222I;
E1*+T2*+A3*+N4*+K5*+S6*+N7*+K8*+Q134L+L150H+N156R+N196W+S199G+N207W+A222I+E284Q;
E96K+D179S+N196W+S199G+A222V+E284Q;
E1*+T2*+A3*+N4*+K5*+S6*+N7*+K8*+E96K+D179S+N196W+S199G+A222V+E284Q;
R64S+E96K+N196W+S199G+N207W+A222V;
N28W+D39R+N196W+S199G+N207W+A222V+E284Q;
D39R+N196W+S199G+A222V+E284Q;
D39R+N196W+S199G+N207W+A222V+E284Q;
E1*+T2*+A3*+N4*+K5*+S6*+N7*+K8*+D39R+N196W+S199G+A222V+E284Q;
E1*+T2*+A3*+N4*+K5*+S6*+N7*+K8*+N196W+N207W+E511D;
L150Y+N196W+S199G+N207W+A222V;
E96K+K101R+L150M+N156R+D179S+N196W+S199G+T208N+A222V;
E96K+K101R+L150M+N156R+D179S+N196W+S199G+T208N+A222V+E284Q;
E1*+T2*+A3*+N4*+K5*+S6*+N7*+K8*+E96K+K101R+L150M+N156R+D179S+N196W+S199G+N207W+T208N+A222V+E284Q;
Q134L+L150H+N156R+N196W+S199G+A222I;
N28W+E96K+D179S+N196W+S199G+A222V+E284Q;
R64S+E96K+N196W+S199G+A222V;
R64S+E96K+N196W+S199G+A222V+E284Q;
E1*+T2*+A3*+N4*+K5*+S6*+N7*+K8*+R64S+E96K+N196W+S199G+A222V;
and wherein the variant has alpha-amylase activity and wherein the variant has at least 60%, at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

10. The process of claim 9, wherein the variant alpha-amylase further comprises a C-terminal deletion, H626*.

11. The process of claim 1, wherein the variant alpha-amylase comprises a combination of alterations selected from:
N28W+H626*;
S199G+H626*;
N196W+V599W+H626*;
N196W+H550Y+P605S+H626*;
N196W+A545P+T576Y+H626*;
N196W+K549Y+G560P+H626*;
I108P+Y183I+N196W+1205Y+H626*;
N196W+R323K+H626*;
N196W+D283P+H626*;
W138Y+N196W+H626*;
L150W+H626*;
N196W+N392W+K417W+H626*;
N196W+N392R+K417W+H626*;
N196W+K549*+H550*+D551*+H626*;
N196W+P580*+E581*+N582*+H626*;
N196W+F592FK+H626*;
N28W+N196W+N207W+S386D+N603W+H626*;
R38Y+N196W+H626*;
N196W+H259Y+H626*;
N196W+Q412W+H626*;
N196W+F212W+H626*;
N196W+V599W+H626*;
N196W+H550Y+P605S+H626*;
N196W+H550Y+K589F+H626*;
N196W+H550Y+D608Y+H626*;
N196W+M574W+L614W+H626*;
N196W+G533H+M574W+L614W+H626*;
N196W+V543P+N570H+H626*;
N196W+G533H+Y575W+L614W+H626*;
N196W+A545P+T576Y+H626*;
N196W+A566P+T578Y+H626*;
N196W+K549Y+G560P+H626*;
N196W+A566P+L577Y+H626*;
N196W+I547Y+G560P+H626*;
N196W+M574MW+H626*;
N196W+K549*+H550*+D551*+M574MW+P580*+E581*+N582*+F592FK+H626*;
N196W+L614W+G619W+H626*;
N28W+I108P+N196W+N207W+S386D+A466V+Q542K+N603W+H626*;
N28W+I108P+N196W+N207W+S386D+N603W+H626*;
N196W+D282P+D283*+H626*;
W138Y+N196W+H626*;
N28W+N196W+N207W+S386D+N603W+H626*;
N196W+S388W+A424P+H626*;
N196W+S388W+A424P+L489Q+H626*;
A117T+N196W+H550Y+D608Y+H626*;
N196W+Q457R+Y575W+L614W+H626*;
N196W+S199G;
N196W+A222V;
N196W+A222E;
N196W+A222I;
S199G+A222V;
S199G+A222E;
S199G+A222I;
N196W+S199G+A222I;
Q134L+H626*;
L150W+N156K+N196W+S199G+A222V;
L150Y+N156K+N196W+S199G+A222I;
L150W+N196W+S199G+A222I+A428S;
L150F+N156K+N196W+S199G+A222I;
L150Y+N156R+N196W+A222V;
L150M+N156R+N196W+S199G+A222I;
L150M+N156R+N196W+A222V;
L150Y+N156R+N196W+S199G+A222V;
L150M+N156K+N196W+S199G+A222V;
L150Y+N156R+N196W+A222I;
L150H+N156R+N196W+S199G+A222I;
L150H+N156K+N196W+A222V;
L150W+N156R+N196W+A222I;
L150F+N156R+N196W+A222I;
L150F+N156K+N196W+S199G+A222V;
L150H+N156K+N196W+S199G+A222V;
L150F+N156R+S199G+A222I;
L150M+N156K+N196W+A222V;
L150W+N156K+N196W+S199G+A222I;
N156K+N196W+S199G+A222V;
L150Y+N156R+S199G+A222I;
L150M+N156R+S199G+A222I;
L150W+N156K+S199G+A222V;
L150W+N156R+N196W+S199G+A222V;
L150Y+N156R+N196W;
N156K+N196W+A222V;
N156K+N196W+S199G;
N156R+S199G+A222V;
S113H+N196W+S199G+A222V;
Q71E+S113H+N196W+S199G+A222V;
N196W+S199G+A222V+D283A;
N196W+S199G+A222V+D283P;
W142E+D193SQY+N196W+S199G+A222V+R224K;
E96K+K101R+L150W+N156R+D179S+N196W+S199G+T208N+A222V;
E96K+K101R+L150Y+N156R+D179S+N196W+S199G+T208N+A222V+E284Q;
E96K+K101R+L150M+N156R+D179S+N196W+S199G+T208N+A222V+E284Q;
S113Q+Q134E+N196W+S199G+A222V;
S113D+Q134N+N196W+S199G+A222V;
S113F+N196W+S199G+A222V;
E171Q+N196W+S199G+N204D+A222V;
N196W+S199G+A222V+H241N+S245N+T278N+E284Q+E285V;
N196W+S199G+A222V+5394K+A414K+K417Y;
N196W+S199G+A222V+E359Y+5394K+K396S+A414K+K417Y;
R38H+E96K+G99N+K101R+D179S+N196W+S199G+A222V;
R38H+E96K+G99N+K101R+D179S+N196W+S199G+A222V+E284Q;
V107T+H110D+N196W+S199G+A222V;
Q134T+L150Y+N196W+S199G+A222V;
S113F+L150W+N196W+S199G+A222V;
S113F+L150Y+N196W+S199G+A222V;
E96K+K101R+N156R+D179S+N196W+S199G+T208N+A222V;
E96K+K101R+L150Y+N156R+D179S+N196W+S199G+T208N+A222V;
E96K+K101R+L150M+N156R+D179S+N196W+S199G+T208N+A222V;
E96K+K101R+N156R+D179S+N196W+S199G+T208N+A222V+E284Q;
E96K+K101R+L150W+N156R+D179S+N196W+S199G+T208N+A222V+E284Q;
N28R+Q86R+N196W+S199G+A222V;
N28R+Q86R+K89R+N196W+S199G+A222V;
G56P+N196W+S199G+S209L+A222V;
E96K+N196W+S199G+A222V;
T10I+N196W+S199G+A222V;
D39R+N196W+S199G+A222V;
R64S+N196W+S199G+A222V;
T10I+D39R+R64S+N196W+S199G+A222V;
T10I+D39R+N196W+S199G+A222V;
D39R+E96K+N196W+S199G+A222V;
R64S+D90E+E96K+N196W+S199G+A222V;
R38H+D39R+E96K+G99N+K101R+D179S+N196W+S199G+A222V;
R38H+R64S+E96K+G99N+K101R+D179S+N196W+S199G+A222V;
T10I+R38H+R64S+E96K+G99N+K101R+D179S+N196W+S199G+A222V;
T10I+R38H+R64S+D90E+E96K+G99N+K101R+D179S+N196W+S199G+A222V;
T10I+R38H+D39R+E96K+G99N+K101R+D179S+N196W+S199G+A222V;
R38H+D39R+R64S+E96K+G99N+K101R+D179S+N196W+S199G+A222V;
E96K+N196W+S199G+A222V+E284Q;
T10I+N196W+S199G+A222V+E284Q;
D39R+N196W+S199G+A222V+E284Q;
R64S+N196W+S199G+A222V+E284Q;
T10I+D39R+E96K+N196W+S199G+A222V+E284Q;
D193SQY+N196W+S199G+A222V;
Q134T+N196W+S199G+A222V;
L174I+N196W+S199G+T208N+A222V;
Y183F+N196W+S199G+T208S+A222V;
N127D+N156R+N196W+S199G+A222V;
Q134T+L150W+N196W+S199G+A222V;
N57P+N196W+S199G+A222V;
N196W+S199G+Q200W+A222V;
T10I+D39R+R64S+E96K+N196W+S199G+A222V+E284Q;
T10I+D39R+R64S+N196W+S199G+A222V+E284Q;
T10I+R64S+E96K+N196W+S199G+A222V+E284Q;
D39R+R64S+D90E+E96K+N196W+S199G+A222V+E284Q;
T10I+R64S+E96K+N196W+S199G+A222V;
T10I+D39R+N196W+S199G+A222V+E284Q;
D39R+E96K+N196W+S199G+A222V+E284Q;
D39R+R64S+N196W+S199G+A222V+E284Q;
R64S+E96K+N196W+S199G+A222V+E284Q;
D39R+R64S+N196W+S199G+A222V;
S12*+S13*+V14*+K15*+N16*+1103Y+N196W+S199G+A222V+N233S+T308Y;
S12*+S13*+V14*+K15*+N16*+A43D+1103Y+N196W+S199G+A222V+N233S+T308M;
V9L+S12P+V14I+N16S+A43T+N196W+S199G+A222V;
S12*+S13*+V14*+K15*+N16*+N196W+S199G+A222V;
N28W+N196W+S199G+A222V;
N196W+S199G+A222V+N392W+K417W;
T10I+D39R+E96K+N196W+S199G+A222V;
V9D+R38H+N196W+S199G+A222V+T348K;
S113F+L150Y+N156K+N196W+S199G+A222V+H626*;
S113Y+L150Y+N156K+N196W+S199G+A222V+H626*;
S113W+L150Y+N156K+N196W+S199G+A222V+H626*;
S113F+N156K+N196W+S199G+A222V+H626*;
S113Y+N156K+N196W+S199G+A222V+H626*;
S113W+N156K+N196W+S199G+A222V+H626*;
W138Y+L150V+N196W+S199G+A222V+H626*;
W138Y+L150V+D179G+N196W+S199G+A222V+H626*;
W138Y+L150V+N196W+S199G+L218W+A222V+H626*;
E96K+Q134L+D179S+N196W+S199G+A222V+E284Q+H626*;
E96K+Q134L+L150Y+N156R+D179S+N196W+S199G+A222V+E284Q+H626*;
E96K+L150Y+N156R+D179S+N196W+S199G+A222V+E284Q+H626*;
R38H+E96K+G99N+K101R+Q134L+D179S+N196W+S199G+S221N+A222V+H626*;
R38H+E96K+G99N+K101R+Q134L+D179S+N196W+S199G+A222V+H626*;
R38H+E96K+G99N+K101R+Q134L+L150Y+N156R+D179S+N196W+S199G+A222V+H626*;
R38H+E96K+G99N+K101R+L150Y+N156R+D179S+N196W+S199G+A222V+H626*;
L150F+N196W+S199G+A222I+H626*;
Q134L+L150F+N156R+N196W+S199G+A222I+H626*;
Q134L+L150H+N156R+N196W+S199G+A222I+H626*;
Q134L+L150M+N156K+N196W+S199G+A222I+H626*;
Q134L+L150Y+N156K+N196W+S199G+A222I+Q457L+H626*;
Q134M+L150W+N156K+N196W+S199G+A222I+H626*;
Q134W+L150W+N156K+N196W+S199G+A222I+H626*;
L150W+L152M+N156K+N196W+S199G+A222I+H626*;
S113F+L150W+N156K+N196W+S199G+A222I+H626*;
S113Y+L150W+N156K+N196W+S199G+A222I+H626*;
L150W+G151W+N156K+N196W+S199G+A222I+H626*;
L150W+G151S+N156K+N196W+S199G+A222I+H626*;
S113F+L150W+G151S+N156K+N196W+S199G+A222I+H626*;
Y67W+W68Y+L150W+N156K+N196W+S199G+A222I+H626*;
A43V+L150M+G151F+N156R+N196W+S199G+A222I;
L150M+G151Y+N156R+N196W+S199G+A222I;
L150M+G151W+N156R+N196W+S199G+A222I;
L150M+G151S+N156R+N196W+S199G+A222I+Y534H;
S113F+L150M+G151S+N156R+N196W+S199G+A222I;
Q134L+L150F+N156K+N196W+S199G+A222I+H626*;
L150W+G151F+N156K+N196W+S199G+A222I+H626*;
L150W+G151Y+N156K+N196W+S199G+A222I+H626*;
Y67W+W68Y+L150W+N156K+N196W+S199G+A222I;
G56W+N57P+Y67W+W68Y+L150W+N156K+N196W+S199G+A222I+H626*;
K54I+Y67W+W68S+S113G+N196W+S199G+A222V+A382T+H626*;
K54I+Y67W+W68S+S113G+D114Q+L150V+N196W+S199G+A222V+H626*;
K54I+Y67W+W68S+S113G+W138Y+N196W+S199G+A222V+H626*;
K54I+Y67W+W68S+S113G+D114Q+W138Y+L150V+N196W+S199G+A222V+H626*;
Q134L+N196W+S199G+A222V+H626*;
V107T+I108L+H110D+F169H+N196W+S199G+A222V+H626*;
N196W+S199G+A222V+N392W+K417W+H626*;
V9D+R38H+N196W+S199G+A222V+T348K+H626*;
E96H+L150Y+N156R+N196W+S199G+A222V+E284Q+H626*;
Q134L+L150Y+N196W+S199G+A222V+H626*;
L150Y+N196W+S199G+A222V+H626*;
Q134L+L150Y+N196W+S199G+A222V+H626*;
L150F+N156R+N196W+S199G+A222V+H626*;
L150H+N156R+N196W+S199G+A222V+H626*;
Q134L+L150F+N156R+N196W+S199G+A222V+H626*;
Q134L+L150H+N156R+N196W+S199G+A222V+H626*;
Q134L+L150F+N156K+N196W+S199G+A222V+H626*;
Q134L+L150H+N156K+N196W+S199G+A222V+H626*;
Q134L+L150Y+N156K+N196W+S199G+A222V+H626*;
L150Y+N156K+N196W+S199G+A222V+H626*;
Q134L+L150Y+N156K+N196W+S199G+A222V+H626*;
Q134W+L150Y+N156K+N196W+S199G+A222V+H626*;
Q134M+L150Y+N156K+N196W+S199G+A222V+H626*;
Q134M+L150Y+N156K+N196W+S199G+A222V+A466V+H626*;
L150Y+L152M+N156K+N196W+S199G+A222V+H626*;
L150S+N196W+S199G+A222V+H626*;
N196W+S199G+A222V+N281S+H626*;
Y67T+N196W+S199G+A222V+H626*;
Q71N+N196W+S199G+A222V+H626*;
Q71N+A94D+N196W+S199G+A222V+H626*;
N196W+S199G+L218F+A222V+H626*;
N196W+S199G+L218W+A222V+H626*;
N196W+S199G+A222V+T278W+H626*;
N196W+S199G+A222V+T278W+T459M+H626*;
N196W+S199G+A222V+T278Y+H626*;
N196W+S199G+A222V+S275N+H626*;
N196W+S199G+A222V+S275L+H626*;
N196W+S199G+A222V+S335Q+H626*;
N196W+S199G+A222V+S335K+H626*;
N196W+S199G+A222V+S335R+H626*;
N196W+S199G+L218W+A222V+S335K+H626*;
N196W+S199G+L218W+A222V+S335Q+H626*;
Y67W+N196W+S199G+A222V+H626*;
N196W+S199G+A222V+N281Q+H626*;
L150M+N156R+N196W+S199G+A222V+H626*;
Q134L+L150M+N156R+N196W+S199G+A222V+H626*;
Q134L+L150M+N156K+N196W+S199G+A222V+H626*;
D39R+N196W+S199G+A222V+N281Q+E284Q;
D39R+N196W+S199G+A222V+E284Q+Q479QP;
D39R+Y70F+N196W+S199G+A222V+E284Q;
N28W+D39R+N196W+S199G+A222V+E284Q;
D39R+N196W+S199G+N207W+A222V+E284Q;
E1*+T2*+A3*+N4*+K5*+S6*+N7*+K8*+D39R+N196W+S199G+N207W+A222V+E284Q+H626*;
N196W+N207W;
N196W+N207W+E284Q;
E1*+T2*+A3*+N4*+K5*+S6*+N7*+K8*+N196W+N207W+E284Q;
L150Y+N196W+S199G+A222V+H626*;
E1*+T2*+A3*+N4*+K5*+S6*+N7*+K8*+L150Y+N196W+S199G+A222V;
E96K+K101R+L150M+N156R+D179S+N196W+S199G+N207W+T208N+A222V;
E1*+T2*+A3*+N4*+K5*+S6*+N7*+K8*+E96K+K101R+L150M+N156R+D179S+N196W+S199G+T208N+A222V;
Q134L+L150H+N156R+N196W+S199G+A222I+E284Q+H626*;
E1*+T2*+A3*+N4*+K5*+S6*+N7*+K8*+Q134L+L150H+N156R+N196W+S199G+A222I+H626*;
E1*+T2*+A3*+N4*+K5*+S6*+N7*+K8*+Q134L+L150H+N156R+N196W+S199G+N207W+A222I+E284Q;
E96K+D179S+N196W+S199G+A222V+E284Q+H626*;
E1*+T2*+A3*+N4*+K5*+S6*+N7*+K8*+E96K+D179S+N196W+S199G+A222V+E284Q;
R64S+E96K+N196W+S199G+N207W+A222V;
N28W+D39R+N196W+S199G+N207W+A222V+E284Q;
D39R+N196W+S199G+A222V+E284Q+H626*;
D39R+N196W+S199G+N207W+A222V+E284Q;
E1*+T2*+A3*+N4*+K5*+S6*+N7*+K8*+D39R+N196W+S199G+A222V+E284Q;
E1*+T2*+A3*+N4*+K5*+S6*+N7*+K8*+N196W+N207W+E511D;
L150Y+N196W+S199G+N207W+A222V;
E96K+K101R+L150M+N156R+D179S+N196W+S199G+T208N+A222V+H626*;
E96K+K101R+L150M+N156R+D179S+N196W+S199G+T208N+A222V+E284Q;
E1*+T2*+A3*+N4*+K5*+S6*+N7*+K8*+E96K+K101R+L150M+N156R+D179S+N196W+S199G+N207W+T208N+A222V+E284Q+H626*;
Q134L+L150H+N156R+N196W+S199G+A222I;
N28W+E96K+D179S+N196W+S199G+A222V+E284Q;
R64S+E96K+N196W+S199G+A222V+H626*;
R64S+E96K+N196W+S199G+A222V+E284Q;
E1*+T2*+A3*+N4*+K5*+S6*+N7*+K8*+R64S+E96K+N196W+S199G+A222V;
and wherein the variant has alpha-amylase activity and wherein the variant has at least 60%, at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

12. The process of claim 14, wherein the variant alpha-amylase comprises combinations of alterations selected from:
S199G+H626*;
N196W+H626*;
N196W+S199G+A222V+H 626*;
N196W+N207W+H626*;
L150Y+N196W+S199G+A222V;
E96K+D179S+N196W+S199G+A222V+E284Q;
R64S+E96K+N196W+S199G+A222V;
and wherein the variant has alpha-amylase activity and wherein the variant has at least 60%, at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1.

13. The process claim 5, wherein the increased pH stability at pH 4.0 can be determined as % residual alpha-amylase activity (% RA) after incubation of the variant amylase at pH 4.0, 32° C., for 18-24 hours.

14. The process claim 5, wherein increased pH stability at pH 4.0 can be determined as residual alpha-amylase activity after incubation of the variant amylase at pH 4.0, 32° C., for 96 hours, and calculation of enzyme half-life in hours.

15. The process of claim 14, wherein half-life is increased compared to the parent amylase of SEQ ID NO: 1, of at least a factor 1.2, 1.3, 1.4, 1.5, 2.0, 2.5, 3.0, 3.5, 4.0, 4.5, 5.0, 6.0, 7.0, such as at least 8.0.

16. The process of claim 1, wherein the variant alpha-amylase further comprises a substitution corresponding to K8N.

17. (canceled)

18. (canceled)

19. (canceled)

20. (canceled)

21. (canceled)

22. (canceled)

23. (canceled)

24. The process of claim 1, wherein steps a) and b) are carried out simultaneously.

25. The process of the claim 1, wherein the fermenting organism is a yeast cell that expresses the variant alpha-amylase of claim 1.

26. (canceled)

27. (canceled)

28. The process of claim 1, wherein the starch-containing material is corn.

29. (canceled)

30. The process of claim 1, wherein the fermentation product is ethanol.


SEQUENCE LISTING (11 sequences)

SEQ ID NO: 1 - protein, 626 aa, Bacillus amyloliquefaciens
ETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPH

SEQ ID NO: 2 - DNA, 1980 bp, Artificial
  Synthetic construct
atgaaaaaaccgctggggaaaattgtcgcaagcaccgcactactcatttctgttgcttttagttcatcgatcgcatcggctgaaactgcaaacaaatcgaataaggtgaccgcgtcatcggtcaaaaacgggaccatcctccatgcatggaattggtcgttcaatacgttaacacaaaatatgaaagatattcgtgatgcgggctatgcagccattcagacgtctccgattaaccaagtaaaggaagggaaccaaggagataaaagcatgaggaactggtactggctgtatcagccgacatcgtaccaaatcggcaaccgttacttaggcactgaacaagaatttaaggacatgtgtgcagccgcggaaaaatatggcgtaaaagtcattgtcgatgcggttatcaatcatactaccagcgattatggcgcgatttctgacgagattaagcgtattccaaactggacccatggaaacacacaaattaaaaattggtcggatcgatgggacgtcactcaaaattcattgcttgggctgtatgattggaatacacagaatactgaggtgcaggcatatctgaaacgtttcttggaaagagcattgaatgacggagcagacgggttccgctatgatgccgccaagcatatagagcttccggatgatgggaattacggcagccaattttggccgaatatcacaaatacatcggcggagttccaatacggagaaatcctgcaagacagcgcgtccagagatacagcttatgcgaattatatgaatgtgacggcttctaactacgggcattccatcagatccgctttaaagaaccgtaatctgagtgtgtcgaatatctcccattatgcatctgacgtgtctgcggacaagttagtcacatgggtggaatcacatgatacgtatgccaatgatgatgaagagtccacatggatgagtgatgacgatattcgtttaggctgggcagtgattggttcccgctcaggaagcacgcctcttttcttttccagacctgagggcggaggaaatggtgtaagatttcccggaaaaagtcaaataggagatcgcgggagcgccttatttaaagatcaggcgatcactgcggtcaatacatttcacaatgtaatggccgggcagcctgaggaactctcgaatccgaatgggaacaaccaaatctttatgaatcagcgcggctcaaaaggcgttgtgctggcaaatgcgggatcgtcttctgtcaccatcaatacttcagcgaaattacctgacggcaggtatgataatagggccggcgccggttcatttcaagtagcgaacggcaaactgacaggtacgatcaatgccagatcggcggctgttctttatcctgatgatattggaaatgcgcctcatgtctttcttgagaattaccaaacgggggcagtccattctttcaatgatcagctgacggtcaccctgcgtgcaaatgcgaaaacaacaaaagccgtttaccaaatcaataatgggcagcagacagcatttaaggatggagaccgactaacgatcgggaaaggagatccaatcggcacgacatacaacatcaaattaaccggaacgaacggcgagggtgcagcgaggacccaagaatacaagtttgttaaaaaagacccgtcccaaaccaacatcattggctatcaaaatccggaccattggggccaggtaaatgcttatatttacaaacatgatggaggcagggccatagaattaaccggatcgtggccggggaaagccatgactaagaatgcagatggaatgtacacgctgacgctgcctgagaatgcggatacggccaacgccaaagtgatttttaacaatggcagcgcccaagtgcccggacagaaccagcccggctttgattatgtgcagaatggtttgtataacaactccggtttaaacggttatcttccgcatcatcatcaccatcaccactaa

SEQ ID NO: 3 - protein, 659 aa, Artificial
  Synthetic construct
MKKPLGKIVASTALLISVAFSSSIASAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPHHHHHHH

SEQ ID NO: 4 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 5 - protein, 583 aa, Artificial
  Synthetic construct
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 6 - protein, 98 aa, Anoxybacillus Contaminans
SQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVP

SEQ ID NO: 7 - protein, 109 aa, Aspergillus kawachii
CTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 8 - protein, 108 aa, Aspergillus niger [sequence omitted]

SEQ ID NO: 9 - protein, 95 aa, Gloeophyllum sepiarium
TVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 10 - protein, 95 aa, Gloeophyllum trabeum
TVAVTFNVNAQTVWGENIYITGSVDALSNWSPDNALLLSSANYPTWSITVNLPASTAIQYKYIRKMNGAVTWESDPNNSITTPASGSWTENDTWR

SEQ ID NO: 11 - protein, 95 aa, Trametes cingulata
TVAVTFNVQATTVFGENIYITGSVPALQNWSPDNALILSAANYPTWSITVNLPASTTIEYKYIRKFNGAVTWESDPNNSITTPASGTFTQNDTWR